SEQUENCE LISTING (30 sequences)

SEQ ID NO: 1 - protein, 5 aa, synthetic construct
  REGION 1..5: note = anti-ILT2 CDRH2
RYYMH

SEQ ID NO: 2 - protein, 17 aa, synthetic construct
  REGION 1..17: note = anti-ILT2 CDRH2
WINPYTGGTNYAQKFQG

SEQ ID NO: 3 - protein, 7 aa, synthetic construct
  REGION 1..7: note = anti-ILT2 CDRH3
KQPSSDY

SEQ ID NO: 4 - protein, 16 aa, synthetic construct
  REGION 1..16: note = anti-ILT2 CDRL1
RSSQSLLHSNTYNYLD

SEQ ID NO: 5 - protein, 7 aa, synthetic construct
  REGION 1..7: note = anti-ILT2 CDRL2
LGSNRAS

SEQ ID NO: 6 - protein, 9 aa, synthetic construct
  REGION 1..9: note = anti-ILT2 CDRL3
LQASQAPWT

SEQ ID NO: 7 - protein, 116 aa, synthetic construct
  REGION 1..116: note = anti-ILT2 VH
QVQLVQSGAEVKKPGASVKVSCKASGYTFNRYYMHWVRQAPGQGLEWMGWINPYTGGTNYAQKFQGRVTMTRDTSISTAYMELSRLRSDDTAVYYCASKQPSSDYWGQGTLVTVSS

SEQ ID NO: 8 - protein, 112 aa, synthetic construct
  REGION 1..112: note = anti-ILT2 VL
DIVMTQSPLSLPVTPGEPASISCRSSQSLLHSNTYNYLDWYLQKPGQSPQLLIYLGSNRASGVPDRFSGSGSGTDFTLKISRVEAEDVGVYYCLQASQAPWTFGQGTKVEIK

SEQ ID NO: 9 - protein, 442 aa, synthetic construct
  REGION 1..442: note = anti-ILT2 Heavy chain
QVQLVQSGAEVKKPGASVKVSCKASGYTFNRYYMHWVRQAPGQGLEWMGWINPYTGGTNYAQKFQGRVTMTRDTSISTAYMELSRLRSDDTAVYYCASKQPSSDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 219 aa, synthetic construct
  REGION 1..219: note = anti-ILT2 Light chain
DIVMTQSPLSLPVTPGEPASISCRSSQSLLHSNTYNYLDWYLQKPGQSPQLLIYLGSNRASGVPDRFSGSGSGTDFTLKISRVEAEDVGVYYCLQASQAPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
  REGION 1..5: note = anti-PD-1 CDRH1
SYGMH

SEQ ID NO: 12 - protein, 17 aa, synthetic construct
  REGION 1..17: note = anti-PD-1 CDRH2
VIWYDGSNKYYADSVKG

SEQ ID NO: 13 - protein, 4 aa, synthetic construct
  REGION 1..4: note = anti-PD-1 CDRH3
NGDH

SEQ ID NO: 14 - protein, 11 aa, synthetic construct
  REGION 1..11: note = anti-PD-1 CDRL1
RASQSVSSNLA

SEQ ID NO: 15 - protein, 7 aa, synthetic construct
  REGION 1..7: note = anti-PD-1 CDRL2
GASTRAT

SEQ ID NO: 16 - protein, 9 aa, synthetic construct
  REGION 1..9: note = anti-PD-1 CDRL3
QQYNNWPRT

SEQ ID NO: 17 - protein, 113 aa, synthetic construct
  REGION 1..113: note = anti-PD-1 VH
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSS

SEQ ID NO: 18 - protein, 107 aa, synthetic construct
  REGION 1..107: note = anti-PD-1 VL
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIK

SEQ ID NO: 19 - protein, 439 aa, synthetic construct
  REGION 1..439: note = anti-PD-1 Heavy chain
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 214 aa, synthetic construct
  REGION 1..214: note = anti-PD-1 Light chain
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, synthetic construct
  REGION 1..5: note = anti-CTLA-4 CDRH1
SYSMN

SEQ ID NO: 22 - protein, 17 aa, synthetic construct
  REGION 1..17: note = anti-CTLA-4 CDRH2
SISSSSSYIYYAESVKG

SEQ ID NO: 23 - protein, 9 aa, synthetic construct
  REGION 1..9: note = anti-CTLA-4 CDRH3
VGLFGPFDI

SEQ ID NO: 24 - protein, 11 aa, synthetic construct
  REGION 1..11: note = anti-CTLA-4 CDRL1
RASQSVSRYLG

SEQ ID NO: 25 - protein, 7 aa, synthetic construct
  REGION 1..7: note = anti-CTLA-4 CDRL2
GASTRAT

SEQ ID NO: 26 - protein, 9 aa, synthetic construct
  REGION 1..9: note = anti-CTLA-4 CDRL3
QQYGSSPWT

SEQ ID NO: 27 - protein, 118 aa, synthetic construct
  REGION 1..118: note = anti-CTLA-4 VH
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSS

SEQ ID NO: 28 - protein, 107 aa, synthetic construct
  REGION 1..107: note = anti-CTLA-4 VL
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIK

SEQ ID NO: 29 - protein, 447 aa, synthetic construct
  REGION 1..447: note = anti-CTLA-4 Heavy chain
EVQLVESGGGLVKPGGSLRLSCAASGFTFSSYSMNWVRQAPGKGLEWVSSISSSSSYIYYAESVKGRFTISRDNAKNSLYLQMNSLRAEDTAVYYCARVGLFGPFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 214 aa, synthetic construct
  REGION 1..214: note = anti-CTLA-4 Light chain
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0347466 A1
(43) Pub. Date: Nov. 5, 2026

(54) METHODS OF TREATING CANCER USING ILT2 ANTIBODIES AND COMBINATION THERAPIES

(71) Applicant: Agenus Inc., Lexington, MA (US)

(72) Inventors: Dhan Sidhartha CHAND, Lexington, MA (US); David Adam SAVITSKY, Lexington, MA (US); Olga UDARTSEVA, Lexington, MA (US)

(21) Appl. No.: 19/565,239

(22) Filed: Mar. 12, 2026

Related U.S. Application Data
(63) Continuation of application No. PCT/US2024/046388, filed on Sep. 12, 2024.
(60) Provisional application No. 63/582,093, filed on Sep. 12, 2023.

(51) Int. Cl. C07K 16/28; A61K 39/00; A61P 35/00; A61P 35/04

(52) U.S. Cl. CPC C07K 16/2803; A61P 35/00; A61P 35/04; C07K 16/2818; C07K 16/2827; A61K 2039/545; C07K 2317/33; C07K 2317/41; C07K 2317/52; C07K 2317/565; C07K 2317/72; C07K 2317/76

(57) ABSTRACT

Provided are methods for treatment of cancer using antibodies that specifically bind to human ILT2, and combinations of these antibodies with human CTLA-4 and/or human PD-1 inhibitors. These methods are particularly advantageous in that the anti-human ILT2 antibodies described herein potently reverse ILT2-mediated immunosuppression, and promote activation of NK, NKT, T cells, and myeloid cells.

### RELATED APPLICATIONS

This application is a continuation application of International Patent Application No. PCT/US2024/046388, filed Sep. 12, 2024, which claims priority to U.S. Provisional Patent Application Ser. No. 63/582,093, filed Sep. 12, 2023, the entire disclosure of each of which is hereby incorporated herein by reference.

### REFERENCE TO SEQUENCE LISTING

This application contains a sequence listing which has been submitted electronically in ST.26 format and is hereby incorporated by reference in its entirety (said ST.26 copy, created on Mar. 12, 2026, is named “361074-2161-SL.xml” and is 40,825 bytes in size).

### FIELD

The present disclosure relates to methods of treating cancer using antibodies that specifically bind to human ILT2, and combination therapies with CTLA-4 and/or PD-1 inhibitors.

### BACKGROUND

ILT2 is an inhibitory receptor belonging to the type I transmembrane glycoproteins, which have four extracellular immunoglobulin-like domains (D1-D4), a transmembrane region, and an intracellular tail with four immunoreceptor tyrosine-based inhibitory motifs (ITIMs). ILT2 is expressed on a variety of immune cells, such as subpopulations of T cells, B cells, natural killer (NK) cells, myeloid-derived suppressive cells (MDSCs), dendritic cells (DCs), and monocytes/macrophages. Additionally, ILT2 binds to classical (HLA-A and -B) and non-classical (HLA-G, HLA-E, and HLA-F) major histocompatibility complex (MHC) class I molecules. High ILT2 expression is associated with poor prognosis in several human malignancies, and ILT2 activation is reported to impair cytotoxic activity of NK and effector T cells, attenuate B cell function, inhibit antigen-presentation by dendritic cells, and promote the immunosuppressive activity of myeloid cells.

CTLA-4 is an inhibitory receptor upregulated on T-cells (Alegre et al., 2001, Nat Rev Immunol 1:220-8). CTLA-4 inhibits the immune response in several ways: it competes with the T cell co-stimulatory receptor CD28 for its ligands, CD80 and CD86, and thus blocks co-stimulation; it negatively signals to inhibit T-cell activation; and it can also capture CD80 and CD86 from opposing cells by trans-endocytosis, resulting in impaired T cell costimulation via CD28 (Krummel and Allison, 1995, J Exp Med 182:459-465; Walunas et al., 1994, Immunity 1:405-413; Qureshi et al., 2011, Science 332:600-603).

PD-1 is another inhibitory receptor that is expressed on activated B cells, T cells, and myeloid cells (Agata et al. (1996) Int Immunol 8:765-72; Okazaki et al. (2002) Curr. Opin. Immunol. 14:391779-82; Bennett et al. (2003) J Immunol 170:711-8). Two ligands for PD-1 have been identified, PD-L1 and PD-L2, that have been shown to downregulate T cell activation upon binding to PD-1 (Freeman et al. (2000) J Exp Med 192:1027-34; Latchman et al. (2001) Nat Immunol 2:261-8; Carter et al. (2002) Eur J Immunol 32:634-43). The interaction between PD-1 and PD-L1/PD-L2 results in a decrease in tumor infiltrating lymphocytes, a decrease in T cell receptor mediated proliferation, and immune evasion by the cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). This immune suppression can be reversed by inhibiting the local interaction of PD-1 with PD-L1 (Iwai et al. (2002) Proc. Nat'l. Acad. Sci. USA 99:12293-7; Brown et al. (2003) J. Immunol. 170:1257-66).

Given the important role of ILT2, CTLA-4, and PD-1 in modulating immune responses, therapies designed to antagonize signaling from these receptors hold great promise for enhancing anticancer immunity and effectively treating cancer.

### SUMMARY

The present disclosure provides methods for treatment of cancer using antibodies that specifically bind to human ILT2, and combinations of these antibodies with human CTLA-4 and/or human PD-1 inhibitors. These methods are particularly advantageous in that the anti-human ILT2 antibodies described herein potently reverse ILT2-mediated immunosuppression, and promote activation of NK, NKT, T cells, and myeloid cells. The therapeutic modulation of ILT2, PD-1, and CTLA-4 co-inhibitory receptors effectively enhances anticancer immunity and overcomes tumor immunosuppression to treat cancer.

Accordingly, in one aspect, the present disclosure provides a method of treating cancer in a subject in need thereof, the method comprising administering to the subject a first antibody that specifically binds to human ILT2 at a dose of about 1 mg to about 2000 mg, wherein the first antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In another aspect, the present disclosure provides a method of treating cancer in a subject in need thereof, the method comprising administering to the subject: a first antibody that specifically binds to human ILT2, wherein the antibody comprises: a VH comprising the CDRH1, CDRH2, and CDRH3 amino acid sequences of the VH amino acid sequence set forth in SEQ ID NO: 7; and a VL comprising the CDRL1, CDRL2, and CDRL3 amino acid sequences of the VL amino acid sequence set forth in SEQ ID NO: 8; and a human PD-1 inhibitor. In certain embodiments, the human PD-1 inhibitor comprises an antagonist antibody or fragment thereof, an inhibitory small molecule, an inhibitory peptide, or inhibitory Fc fusion protein.

In another aspect, the present disclosure provides a method of treating cancer in a subject in need thereof, the method comprising administering to the subject: a first antibody that specifically binds to human ILT2, wherein the antibody comprises: a VH comprising the CDRH1, CDRH2, and CDRH3 amino acid sequences of the VH amino acid sequence set forth in SEQ ID NO: 7; and a VL comprising the CDRL1, CDRL2, and CDRL3 amino acid sequences of the VL amino acid sequence set forth in SEQ ID NO: 8; and a human CTLA-4 inhibitor. In certain embodiments, the human CTLA-4 inhibitor comprises an antagonist antibody or fragment thereof, an inhibitory small molecule, an inhibitory peptide, or inhibitory Fc fusion protein.

In another aspect, the present disclosure provides a method of treating cancer in a subject in need thereof, the method comprising administering to the subject: a first antibody that specifically binds to human ILT2, wherein the antibody comprises: a VH comprising the CDRH1, CDRH2, and CDRH3 amino acid sequences of the VH amino acid sequence set forth in SEQ ID NO: 7; and a VL comprising the CDRL1, CDRL2, and CDRL3 amino acid sequences of the VL amino acid sequence set forth in SEQ ID NO: 8, and a human PD-L1 inhibitor. In certain embodiments, the human PD-L1 inhibitor comprises an antagonist antibody or fragment thereof, an inhibitory small molecule, an inhibitory peptide, or inhibitory Fc fusion protein.

In another aspect, the present disclosure provides a method of treating cancer in a subject in need thereof, the method comprising administering to the subject: a first antibody that specifically binds to human ILT2, wherein the antibody comprises: a VH comprising the CDRH1, CDRH2, and CDRH3 amino acid sequences of the VH amino acid sequence set forth in SEQ ID NO: 7; and a VL comprising the CDRL1, CDRL2, and CDRL3 amino acid sequences of the VL amino acid sequence set forth in SEQ ID NO: 8, and a human PD-L2 inhibitor. In certain embodiments, the human PD-L2 inhibitor comprises an antagonist antibody or fragment thereof, an inhibitory small molecule, an inhibitory peptide, or inhibitory Fc fusion protein.

In another aspect, the present disclosure provides a method of treating cancer in a subject in need thereof, the method comprising administering to the subject: a first antibody that specifically binds to human ILT2, wherein the antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8; a human PD-1 inhibitor; and a human CTLA-4 inhibitor.

In certain embodiments, the first antibody is administered at a dose of about 1 mg to about 1000 mg. In certain embodiments, the first antibody is administered at a dose of about 1 mg, about 3 mg, about 10 mg, about 30 mg, about 100 mg, about 300 mg, or about 1000 mg.

In certain embodiments, the first antibody is administered intravenously. In certain embodiments, the first antibody is administered by intravenous infusion over about 30 minutes.

In certain embodiments, the first antibody is administered once weekly. In certain embodiments, the first antibody is administered once every 2 weeks. In certain embodiments, the first antibody is administered once every 3 weeks. In certain embodiments, the first antibody is administered once every 4 weeks. In certain embodiments, the first antibody is administered once every 5 weeks. In certain embodiments, the first antibody is administered once every 6 weeks.

In certain embodiments, the first antibody is administered intravenously at a dose of 1 mg once every 3 weeks. In certain embodiments, the first antibody is administered intravenously at a dose of 3 mg once every 3 weeks. In certain embodiments, the first antibody is administered intravenously at a dose of 10 mg once every 3 weeks. In certain embodiments, the first antibody is administered intravenously at a dose of 30 mg once every 3 weeks. In certain embodiments, the first antibody is administered intravenously at a dose of 100 mg once every 3 weeks. In certain embodiments, the first antibody is administered intravenously at a dose of 300 mg once every 3 weeks. In certain embodiments, the first antibody is administered intravenously at a dose of 1000 mg once every 3 weeks.

In certain embodiments, the cancer is a solid tumor. In certain embodiments, the solid tumor is metastatic or locally advanced. In certain embodiments, the cancer is selected from the group consisting of bladder cancer, brain cancer, breast cancer, cervical cancer, colon and/or rectal cancer, endometrial cancer, head and neck cancer, kidney cancer, liver cancer, melanoma, mesothelioma, non-small cell lung cancer, oral cancer, ovarian cancer, pancreatic cancer, prostate cancer, sarcoma, skin cancer, small cell lung cancer, stomach cancer, and thyroid cancer.

In certain embodiments, before administration of the first antibody: the subject is at least 18 years of age; the subject has a histologically confirmed diagnosis of a solid tumor that is currently metastatic or locally advanced, wherein no standard therapy is available or standard therapy has failed; the subject has measurable disease on baseline imaging as assessed according to Response Evaluation Criteria in Solid Tumors Version 1.1 (RECIST 1.1); the subject has a predicted life expectancy of ≥3 months; the subject has an Eastern Cooperative Oncology Group performance status of 0-1; the subject has adequate organ and bone marrow reserve function as defined by one or more of: absolute neutrophil count ≥1.5×10⁹/L; platelet count ≥100×10⁹/L; hemoglobin ≥8.0 g/dL without transfusion within 2 weeks prior to hemoglobin measurement; serum albumin ≥3.0 g/dL; total bilirubin ≤1.5× upper limit of normal (ULN); aspartate aminotransferase ≤2.5×ULN; alanine aminotransferase ≤2.5×ULN; alkaline phosphatase ≤2.5×ULN, or ≤5.0×ULN for subjects with liver metastases; creatinine clearance ≥40 mL/min as assessed by Cockcroft-Gault method; and/or international normalized ratio or prothrombin time ≤1.5×ULN and activated partial thromboplastin time ≤1.5×ULN, unless patient is receiving anticoagulant therapy; the subject is not pregnant and/or breastfeeding; the subject has not had a cytotoxic therapy or other monoclonal antibody therapy within 3 weeks prior; the subject has not had radiation therapy to the central nervous system (CNS) within 2 weeks prior or non-CNS radiation therapy within one week prior; and/or the subject has not had small molecule targeted therapy or tyrosine kinase inhibitor therapy within 2 weeks prior or 5 half-lives; the subject does not have any persistent toxicities (Common Terminology Criteria for Adverse Events [CTCAE]≥1) from prior cancer therapies; the subject does not have sensory neuropathy or alopecia of Grade ≥2; the subject does not have a history of or active interstitial lung disease; the subject does not have a history of anaphylaxis or uncontrolled asthma; the subject does not have a condition requiring systemic treatment with either corticosteroids (>10 mg daily prednisone equivalent) within 2 weeks prior or other immunosuppressive medication within 30 days prior; the subject does not have active CNS metastases, unless the CNS metastases has been treated and the subject is radiologically and clinically stable; the subject does not have active or history of autoimmune disease that requires systemic treatment within 2 years prior, unless treatment consists of hormone replacement therapy or topical treatment; the subject has not had an allogeneic tissue or solid organ transplant, except for a corneal transplant; the subject has not had an active infection requiring treatment within 2 weeks prior; the subject is not HIV positive, unless the subject has CD4>200 cells/mm³ or an undetectable viral load; the subject does not have active hepatitis B (HBV) or hepatitis C (HPC), unless hepatitis B surface antigen and HBV DNA, or HCV RNA, respectively, are negative; the subject does not have clinically significant cardiovascular disease, as defined by one or more of: cerebral vascular accident or stroke or myocardial infarction within 6 months prior; unstable angina; congestive heart failure (New York Heart Association class ≥II); and/or uncontrolled cardiac arrhythmia requiring medication; the subject does not have QTc interval >480 msec, unless prolonged QTc is due to right bundle branch block; and/or the subject does not have uncontrolled hypertension (>140/90 mmHg), or controlled hypertension on more than 3 antihypertensive agents.

In certain embodiments, the first antibody comprises the CDRH1, CDRH2, CDRH3, CDRL1, CDRL2, and CDRL3 amino acid sequences, set forth in SEQ ID NOs: 1, 2, 3, 4, 5, and 6, respectively. In certain embodiments, the first antibody comprises the VH amino acid sequence of SEQ ID NO: 7, and/or the VL amino acid sequence of SEQ ID NO: 8. In certain embodiments, the first antibody comprises the VH and VL amino acid sequences of SEQ ID NOs: 7 and 8, respectively. In certain embodiments, the first antibody comprises a heavy chain constant region selected from the group consisting of human IgG₁, IgG₂, IgG₃, IgG₄, IgA₁, IgA₂, and IgM. In certain embodiments, the heavy chain constant region is a human IgG₄ heavy chain constant region comprising a P at position 228, numbered according to the EU numbering system. In certain embodiments, the first antibody comprises a heavy chain comprising the amino acid sequence of SEQ ID NO: 9, and/or a light chain comprising the amino acid sequence of SEQ ID NO: 10. In certain embodiments, the first antibody comprises a heavy chain and a light chain comprising the amino acid sequences of SEQ ID NOs: 9 and 10, respectively.

In certain embodiments, the human PD-1 inhibitor is a second antibody that specifically binds to human PD-1, human PD-L1, or human PD-L2. In certain embodiments, the second antibody comprises: a VH comprising the CDRH1, CDRH2, and CDRH3 amino acid sequences of the VH amino acid sequence set forth in SEQ ID NO: 17; and a VL comprising the CDRL1, CDRL2, and CDRL3 amino acid sequences of the VL amino acid sequence set forth in SEQ ID NO: 18. In certain embodiments, the second antibody comprises the CDRH1, CDRH2, CDRH3, CDRL1, CDRL2, and CDRL3 amino acid sequences, set forth in SEQ ID NOs: 11, 12, 13, 14, 15, and 16, respectively. In certain embodiments, the second antibody comprises the VH amino acid sequence of SEQ ID NO: 17, and/or the VL amino acid sequence of SEQ ID NO: 18. In certain embodiments, the second antibody comprises the VH and VL amino acid sequences of SEQ ID NOs: 17 and 18, respectively. In certain embodiments, the second antibody comprises a heavy chain constant region selected from the group consisting of human IgG₁, IgG₂, IgG₃, IgG₄, IgA₁, IgA₂, and IgM. In certain embodiments, the heavy chain constant region is a human IgG₄ heavy chain constant region comprising a P at position 228, numbered according to the EU numbering system. In certain embodiments, the second antibody comprises a heavy chain comprising the amino acid sequence of SEQ ID NO: 19, and/or a light chain comprising the amino acid sequence of SEQ ID NO: 20. In certain embodiments, the second antibody comprises a heavy chain and a light chain comprising the amino acid sequences of SEQ ID NOs: 19 and 20, respectively.

In certain embodiments, the second antibody is selected from the group consisting of balstilimab, adebrelimab, atezolizumab, avelumab, camrelizumab, cemiplimab, cosibelimab, dostarlimab, durvalumab, enlonstobart, envafolimab, nivolumab, pembrolizumab, penpulimab, pidilizumab, prolgolimab, pucotenlimab, retifanlimab, serplulimab, sintilimab, socazolimab, sugemalimab, tagitanlimab, tislelizumab, toripalimab, and zimberelimab.

In certain embodiments, the second antibody is administered at a dose of about 450 mg. In certain embodiments, the second antibody is administered at a dose of about 3 mg/kg.

In certain embodiments, the second antibody is administered by intravenous infusion over about 30 minutes. In certain embodiments, the second antibody is administered once weekly. In certain embodiments, the second antibody is administered once every 2 weeks. In certain embodiments, the second antibody is administered once every 3 weeks. In certain embodiments, the second antibody is administered to the subject subsequent to administration of the first antibody.

In certain embodiments, the human CTLA-4 inhibitor is a third antibody that specifically binds human CTLA-4. In certain embodiments, the third antibody comprises: a VH comprising the CDRH1, CDRH2, and CDRH3 amino acid sequences of the VH amino acid sequence set forth in SEQ ID NO: 27; and a VL comprising the CDRL1, CDRL2, and CDRL3 amino acid sequences of the VL amino acid sequence set forth in SEQ ID NO: 28. In certain embodiments, the third antibody comprises the CDRH1, CDRH2, CDRH3, CDRL1, CDRL2, and CDRL3 amino acid sequences, set forth in SEQ ID NOs: 21, 22, 23, 24, 25, and 26, respectively. In certain embodiments, the third antibody comprises the VH amino acid sequence of SEQ ID NO: 27, and/or the VL amino acid sequence of SEQ ID NO: 28. In certain embodiments, the third antibody comprises the VH and VL amino acid sequences of SEQ ID NOs: 27 and 28, respectively. In certain embodiments, the third antibody comprises a heavy chain constant region selected from the group consisting of human IgG₁, IgG₂, IgG₃, IgG₄, IgA₁, IgA₂, and IgM. In certain embodiments, the third antibody comprises a human IgG₁ heavy chain constant region that is a variant of a wild type human IgG₁ heavy chain constant region, wherein the variant human IgG₁ heavy chain constant region binds to FcγRIIIA with a higher affinity as compared to the affinity that the wild type human IgG₁ heavy chain constant region binds to FcγRIIIA. In certain embodiments, the third antibody is afucosylated. In certain embodiments, the heavy chain constant region is a human IgG₁ heavy chain constant region comprising a D at position 239, a L at position 330, and an E at position 332, numbered according to the EU numbering system. In certain embodiments, the third antibody comprises a heavy chain comprising the amino acid sequence of SEQ ID NO: 29, and/or a light chain comprising the amino acid sequence of SEQ ID NO: 30. In certain embodiments, the third antibody comprises a heavy chain and a light chain comprising the amino acid sequences of SEQ ID NOs: 29 and 30, respectively. In certain embodiments, the third antibody is selected from the group consisting of botensilimab, ipilimumab, and tremelimumab.

In certain embodiments, the third antibody is administered at a dose of about 150 mg. In certain embodiments, the third antibody is administered at a dose of about 75 mg. In certain embodiments, the third antibody is administered at a dose of about 50 mg. In certain embodiments, the third antibody is administered at a dose of about 2 mg/kg. In certain embodiments, the third antibody is administered at a dose of about 4 mg/kg.

In certain embodiments, the third antibody is administered by intravenous infusion over about 30 minutes. In certain embodiments, the third antibody is administered once every 2 weeks. In certain embodiments, the third antibody is administered once every 3 weeks. In certain embodiments, the third antibody is administered once every 4 weeks. In certain embodiments, the third antibody is administered once every 6 weeks.

In certain embodiments, the third antibody is administered to the subject subsequent to administration of the first antibody. In certain embodiments, the third antibody is administered to the subject subsequent to administration of the first antibody and second antibody.

In another aspect, the present disclosure provides a method of treating cancer in a subject in need thereof, the method comprising administering to the subject: a first antibody that specifically binds to human ILT2, wherein the antibody comprises a heavy chain and a light chain comprising the amino acid sequences of SEQ ID NOs: 9 and 10, respectively; balstilimab at a dose of 450 mg once every 3 weeks by intravenous infusion; and botensilimab at a dose of 150 mg once every 6 weeks by intravenous infusion.

In certain embodiments, the first antibody is administered at a dose of about 1 mg to about 1000 mg. In certain embodiments, the first antibody is administered at a dose of about 1 mg, about 3 mg, about 10 mg, about 30 mg, about 100 mg, about 300 mg, or about 1000 mg.

In certain embodiments, the first antibody is administered intravenously. In certain embodiments, the first antibody is administered by intravenous infusion over about 30 minutes.

In certain embodiments, the first antibody is administered once weekly. In certain embodiments, the first antibody is administered once every 2 weeks. In certain embodiments, the first antibody is administered once every 3 weeks. In certain embodiments, the first antibody is administered once every 4 weeks. In certain embodiments, the first antibody is administered once every 5 weeks. In certain embodiments, the first antibody is administered once every 6 weeks.

In certain embodiments, the first antibody is administered intravenously at a dose of 1 mg once every 3 weeks. In certain embodiments, the first antibody is administered intravenously at a dose of 3 mg once every 3 weeks. In certain embodiments, the first antibody is administered intravenously at a dose of 10 mg once every 3 weeks. In certain embodiments, the first antibody is administered intravenously at a dose of 30 mg once every 3 weeks. In certain embodiments, the first antibody is administered intravenously at a dose of 100 mg once every 3 weeks. In certain embodiments, the first antibody is administered intravenously at a dose of 300 mg once every 3 weeks. In certain embodiments, the first antibody is administered intravenously at a dose of 1000 mg once every 3 weeks.

In certain embodiments, the cancer is a solid tumor. In certain embodiments, the solid tumor is metastatic or locally advanced. In certain embodiments, the cancer is selected from the group consisting of bladder cancer, brain cancer, breast cancer, cervical cancer, colon and/or rectal cancer, endometrial cancer, head and neck cancer, kidney cancer, liver cancer, melanoma, mesothelioma, non-small cell lung cancer, oral cancer, ovarian cancer, pancreatic cancer, prostate cancer, sarcoma, skin cancer, small cell lung cancer, stomach cancer, and thyroid cancer.

In another aspect, the present disclosure provides an antibody that specifically binds to human ILT2 for use in the treatment of cancer, wherein the treatment is performed according to the method of any one of the preceding claims.

In another aspect, the present disclosure provides an antibody that specifically binds to human ILT2 for use in the manufacture of a medicament for the treatment of cancer, wherein the treatment is performed according to the method of any one of the preceding claims.

In another aspect, the present disclosure provides use of an antibody that specifically binds to human ILT-2 for the treatment of cancer, wherein the treatment is performed according to the method of any one of the previous claims.

### BRIEF DESCRIPTION OF THE DRAWINGS

FIGS. 1A-1D depict exemplary data of the additive effect of the combination of anti-ILT-2 AGEN1571 and anti-PD-1 AGEN2034 (balstilimab) on immune cell activation. In brief, PBMCs were primed with IL-2 and IL-15 in presence of (i) AGEN1571, (ii) AGEN2034, (iii) AGEN1571+AGEN2034 combination, or (iv) isotype control antibodies followed by co-cultivation with HLA-G expressing JEG-3^{HLA-A2} tumor cells for 72 h. Frequencies of CD25⁺ on NK (FIG. 1A), NKT (FIG. 1B), and CD8⁺ T cells (FIG. 1C) were detected by flow cytometry and plotted using GraphPad Prism. FMO controls were used to set up gating. Data for 10 donors tested in 3 independent experiments are shown. Luminex immunoassay was used to profile TNF alpha (FIG. 1D), IFN gamma (FIG. 1E), and MIP-1 beta (FIG. 1F). Statistical analysis was performed by one-way ANOVA test. P-values <0.05 were considered statistically significant to denominate difference between groups (*p<0.05, ** p<0.01, *** p<0.001, **** p<0.0001).

FIGS. 2A-2B depict exemplary dose-response curves illustrating the additive effect of the combination of anti-ILT2 AGEN1571 and anti-CTLA-4 AGEN1181 (botensilimab). In brief, engineered Jurkat ILT2 CD16 NFAT luciferase cells were co-cultured with Raji-CTLA-4 cells in the presence of AGEN1181 and AGEN1571. Identical data are plotted in FIGS. 2A and 2B with AGEN1181 dose titration shown on the x-axis (FIG. 2A) or AGEN1571 titration shown on the x-axis (FIG. 2B). IgG₄ and IgG₁.DLE isotype controls are also shown. Luciferase activity from the NFAT reporter gene induced by the activity of Fc gamma receptor IIIA (CD16) was measured and expressed as relative luminesce unit (RLU).

### DETAILED DESCRIPTION

The present disclosure provides methods for treatment of cancer using antibodies that specifically bind to human ILT2, and combinations of these antibodies with human CTLA-4 and/or human PD-1 inhibitors. These methods are particularly advantageous in that the anti-human ILT2 antibodies described herein are potent blockers of ILT2-HLA-G interactions and have been shown to enhance immune cell function, including increased activation of macrophages. The therapeutic modulation of ILT2, PD-1, and CTLA-4 co-inhibitory receptors effectively enhances anticancer immunity and overcomes tumor immunosuppression to treat cancer.

As used herein, the terms “about” and “approximately,” when used to modify a numeric value or numeric range, indicate that deviations of 5% to 10% above and 5% to 10% below the value or range remain within the intended meaning of the recited value or range.

As used herein, “ILT2,” refers to immunoglobulin-like transcript 2, which is also known as leukocyte immunoglobulin-like receptor subfamily B member 1 (LILRB1) or leukocyte immunoglobulin-like receptor 1 (LIR-1). As used herein, the term “human ILT2” refers to a human ILT2 protein encoded by a wild-type ILT2 gene, e.g., RefSeq accession number NC_000019.10. An exemplary immature amino acid sequence of human ILT2 is provided as in RefSeq accession number NP_006660.4.

As used herein, the term “CTLA-4” refers to cytotoxic T-lymphocyte-associated protein 4. As used herein, the term “human CTLA-4” refers to a human CTLA-4 protein encoded by a wild-type human CTLA-4 gene, e.g., RefSeq accession number NM_005214.5 or NM_001037631.2. An exemplary immature amino acid sequence of human CTLA-4 is set forth in RefSeq accession number NP_005205.2.

As used herein, the term “PD-1” refers to programmed cell death protein 1. As used herein, the term “human PD-1” refers to a human PD-1 protein encoded by a wild-type human PD-1 gene, e.g., RefSeq accession number NM_005018.3. An exemplary immature amino acid sequence of human PD-1 is provided as in RefSeq accession number NP_005009.2.

As used herein, the term “PD-L1” refers to the programmed cell death ligand 1. As used herein, the term “human PD-L1” refers to a human PD-L1 protein encoded by a wild-type human PD-L1 gene, e.g., RefSeq accession number NM_014143.4. An exemplary immature amino acid sequence of human PD-L1 is provided as in RefSeq accession number NP_054862.1.

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single-chain antibodies or single-chain Fvs (scFv), camelized antibodies, affibodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG₂b) of immunoglobulin molecule. In certain embodiments, antibodies described herein are IgG antibodies, or a class (e.g., human IgG₁ or IgG₄) or subclass thereof. In a specific embodiment, the antibody is a humanized antibody. In another specific embodiment, the antibody is a human antibody.

As used herein, the term “CDR” or “complementarity determining region” means the noncontiguous antigen combining sites found within the variable regions of heavy and light chain polypeptides. In certain embodiments, CDRs of an antibody disclosed herein are determined according to Kabat et al., J. Biol. Chem. 252, 6609-6616 (1977) and Kabat et al, Sequences of protein of immunological interest (1991), each of which is herein incorporated by reference in its entirety. In certain embodiments, CDRs of an antibody disclosed herein are determined according to the Chothia numbering scheme (see, e.g., Chothia C & Lesk A M, (1987), J Mol Biol 196:901-917; Al-Lazikani B et al., (1997) J Mol Biol 273:927-948; Chothia C et al., (1992) J Mol Biol 227:799-817; Tramontano A et al., (1990) J Mol Biol 215(1): 175-82; and U.S. Pat. No. 7,709,226, all of which are herein incorporated by reference in their entireties). In certain embodiments, CDRs of an antibody disclosed herein are determined according to MacCallum R M et al., (1996) J Mol Biol 262:732-745, herein incorporated by reference in its entirety. See also, e.g., Martin A. “Protein Sequence and Structure Analysis of Antibody Variable Domains,” in Antibody Engineering, Kontermann and Dribel, eds., Chapter 31, pp. 422-439, Springer-Verlag, Berlin (2001), herein incorporated by reference in its entirety. In certain embodiments, CDRs of an antibody disclosed herein are determined according to the IMGT numbering system as described in: Lefranc M-P, (1999) The Immunologist 7:132-136; Lefranc M-P et al, (1999) Nucleic Acids Res 27:209-212, each of which is herein incorporated by reference in its entirety; and Lefranc M-P et al, (2009) Nucleic Acids Res 37: D1006-D1012. In certain embodiments, CDRs of an antibody disclosed herein are determined according to the AbM numbering scheme, which refers to AbM hypervariable regions, which represent a compromise between the Kabat CDRs and Chothia structural loops and are used by Oxford Molecular's AbM antibody modeling software (Oxford Molecular Group, Inc.), herein incorporated by reference in its entirety. In certain embodiments, CDRs of an antibody disclosed herein are determined according to the AHo numbering system, as described in Honegger and Pluckthun, J. Mol. Biol. 309:657-670 (2001), herein incorporated by reference in its entirety. In certain embodiments, CDRs of an antibody disclosed herein are each independently determined according to one of the Kabat, Chothia, MacCallum, IMGT, AHo, or AbM numbering schemes, or by structural analysis of the multispecific molecule, wherein the structural analysis identifies residues in the variable region(s) predicted to make contact with an epitope. CDRH1, CDRH2, and CDRH3 denote the heavy chain CDRs, and CDRL1, CDRL2, and CDRL3 denote the light chain CDRs.

As used herein, the terms “VH” and “VL” refer to antibody heavy and light chain variable regions, respectively, as described in Kabat et al., (1991) Sequences of Proteins of Immunological Interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen but which can exhibit various effector functions, such as interaction with an Fc receptor (e.g., Fc gamma receptor).

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ), based on the amino acid sequence of the constant region. Light chain amino acid sequences are well known in the art. In specific embodiments, the light chain is a human light chain.

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991.

As used herein, the term “specifically binds” refers to the specificity of an antibody for an antigen, as is understood by one skilled in the art. Binding molecules that specifically bind to an antigen typically bind to the antigen with an equilibrium dissociation constant (KD) of less than 1×10 6 M, as measured by, e.g., ELISA assay, surface plasmon resonance, or other suitable assays known in the art. The skilled worker will appreciate that, in certain embodiments, a binding molecule can specifically bind to different antigens, e.g., different antigens that share a common epitope that is recognized by the binding molecule.

As used herein, the term “afucosylated” in the context of an Fc refers to a substantial lack of a fucose covalently attached, directly or indirectly, to residue 297 of the human IgG₁ Fc region, numbered according to the EU numbering system, or the corresponding residue in non-IgG₁ or non-human IgG₁ immunoglobulins. Thus, in a composition comprising a plurality of afucosylated antibodies, at least 70% of the antibodies will not be fucosylated, directly or indirectly (e.g., via intervening sugars) at residue 297 of the Fc region of the antibodies, and in some embodiments at least 80%, 85%, 90%, 95%, or 99% will not be fucosylated, directly or indirectly, at residue 297 of the Fc region.

As used herein, the term “CTLA-4 inhibitor” refers to a molecule that can inhibit the binding of CTLA-4 to its ligand, Cluster of differentiation 80 (CD80).

As used herein, the term “PD-1 inhibitor” refers to a molecule that can inhibit the binding of PD-1 to its ligand, Programmed death-ligand 1 (PD-L1).

As used herein, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251:6300-6303). Antibody: antigen crystals may be studied using well known X-ray diffraction techniques and may be refined using computer software such as X-PLOR (Yale University, 1992, distributed by Molecular Simulations, Inc.; see e.g. Meth Enzymol (1985) volumes 114 & 115, eds Wyckoff H W et al.; U.S. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323). Mutagenesis mapping studies may be accomplished using any method known to one of skill in the art. See, e.g., Champe M et al., (1995) J Biol Chem 270:1388-1394 and Cunningham B C & Wells J A (1989) Science 244:1081-1085 for a description of mutagenesis techniques, including alanine scanning mutagenesis techniques. In a specific embodiment, the epitope of an antibody is determined using alanine scanning mutagenesis studies.

As used herein, the term “treat,” “treating,” and “treatment” refer to therapeutic or preventative measures described herein. The methods of “treatment” employ administration of an antibody to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “therapeutic combination” refers to the combination of a first therapy and the second therapy administered to a subject. The first therapy and the second therapy can be administered simultaneously (in the same pharmaceutical composition or in separate pharmaceutical compositions) or sequentially in any order.

As used herein, the term “effective amount” in the context of the administration of a therapy to a subject refers to the amount of a therapy that achieves a desired prophylactic or therapeutic effect. The effective amount of a therapeutic combination of a first therapy and a second therapy includes a first amount of the first therapy and a second amount of the second therapy, wherein the administration of the therapeutic combination achieves a desired prophylactic or therapeutic effect.

As used herein, the term “subject” includes any human or non-human animal. In certain embodiments, the subject is a human or non-human mammal. In certain embodiments, the subject is a human.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F (1990) PNAS 87:2264-2268, modified as in Karlin S & Altschul S F (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “Eastern Cooperative Oncology Group performance status” refers to the grade on the Eastern Cooperative Oncology Group Performance Status Scale determined for a subject prior to treatment. The Eastern Cooperative Oncology Group Performance Status Scale is well known in the art and describes a patient's level of function in terms of ability to care for themself, daily activity, and physical ability (walking, working, etc.).

As used herein, the term “Cockcroft-Gault method” refers to a method of calculating creatinine clearance (CrCl) from serum creatinine, age, and weight of a subject. The method is well known in the art and described in Cockcroft D W, Gault M H. Nephron. 1976; 16(1):31-41), which is herein incorporated by reference in its entirety.

As used herein, the terms “Response Evaluation Criteria in Solid Tumors Version 1.1” and “RECIST 1.1” refer to a standardized method of radiological imaging to measure the response of a tumor to treatment. RECIST 1.1 is well known in the art and described in, e.g., Therasse P, et al. JNCI. 2000; 92(3):205-216.

### Anti-ILT2 Antibodies

Anti-human ILT2 antibodies that are useful in the methods and uses described herein include, but are not limited to, those described below.

In certain embodiments, the antibody that specifically binds to human ILT2 comprises: a heavy chain variable region (VH) comprising the CDRH1, CDRH2, and CDRH3 amino acid sequences of the VH amino acid sequence set forth in SEQ ID NO: 7. In certain embodiments, the antibody that specifically binds to human ILT2 comprises: a light chain variable region (VL) comprising the CDRL1, CDRL2, and CDRL3 amino acid sequences of the VL amino acid sequence set forth in SEQ ID NO: 8. In certain embodiments, the antibody comprises a heavy chain variable region (VH) comprising the CDRH1, CDRH2, and CDRH3 amino acid sequences of the VH amino acid sequence set forth in SEQ ID NO: 7 and a light chain variable region (VL) comprising the CDRL1, CDRL2, and CDRL3 amino acid sequences of the VL amino acid sequence set forth in SEQ ID NO: 8.

In certain embodiments, the antibody that specifically binds to human ILT2 comprises the CDRH1, CDRH2, and CDRH3 amino acid sequences set forth in SEQ ID NOs: 1, 2, and 3, respectively. In certain embodiments, the antibody that specifically binds to human ILT2 comprises the CDRL1, CDRL2, and CDRL3 amino acid sequences set forth in SEQ ID NOs: 4, 5, and 6, respectively. In certain embodiments, the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

In certain embodiments, the antibody that specifically binds to human ILT2 comprises a VH comprising an amino acid sequence which is at least 90%, 95%, 96%, 97%, 98%, 99%, or 100% identical to the amino acid sequence set forth in SEQ ID NO: 7. In certain embodiments, the antibody comprises a VH comprising the amino acid sequence set forth in SEQ ID NO: 7.

In certain embodiments, the antibody that specifically binds to human ILT2 comprises a VL comprising an amino acid sequence which is at least 90%, 95%, 96%, 97%, 98%, 99%, or 100% identical to the amino acid sequence set forth in SEQ ID NO: 8. In certain embodiments, the antibody comprises a VL comprising the amino acid sequence set forth in SEQ ID NO: 8.

In certain embodiments, the antibody that specifically binds to human ILT2 comprises: a VH comprising an amino acid sequence which is at least 90%, 95%, 96%, 97%, 98%, 99%, or 100% identical to the amino acid sequence set forth in SEQ ID NO: 7; and a VL comprising an amino acid sequence which is at least 90%, 95%, 96%, 97%, 98%, 99%, or 100% identical to the amino acid sequence set forth in SEQ ID NO: 8. In certain embodiments, the antibody comprises a VH comprising the amino acid sequence set forth in SEQ ID NO: 7 and a VL comprising the amino acid sequence set forth in SEQ ID NO: 8.

In certain embodiments, the antibody that specifically binds to human ILT2 comprises a heavy chain constant region selected from the group consisting of human IgG₁, IgG₂, IgG₃, IgG₄, IgA₁, and IgA₂. In certain embodiments, the heavy chain constant region is IgG₄. In certain embodiments, the antibody comprises a light chain constant region selected from the group consisting of a human kappa light chain constant region and a human lambda light chain constant region.

In certain embodiments, the antibody that specifically binds to human ILT2 comprises a heavy chain constant region, e.g., an IgG₄ constant region, or fragment thereof comprising an S228P mutation, numbered according to the EU numbering system. In certain embodiments, the heavy chain constant region is a human IgG₄ heavy chain constant region comprising a P at position 228, numbered according to the EU numbering system.

In certain embodiments, the antibody that specifically binds to human ILT2 comprises a heavy chain comprising an amino acid sequence which is at least 90%, 95%, 96%, 97%, 98%, 99%, or 100% identical to the amino acid sequence set forth in SEQ ID NO: 9. In certain embodiments, the antibody comprises a heavy chain comprising the amino acid sequence set forth in SEQ ID NO: 9.

In certain embodiments, the antibody that specifically binds to human ILT2 comprises a light chain comprising an amino acid sequence which is at least 90%, 95%, 96%, 97%, 98%, 99%, or 100% identical to the amino acid sequence set forth in SEQ ID NO: 10. In certain embodiments, the antibody comprises a light chain comprising the amino acid sequence set forth in SEQ ID NO: 10.

In certain embodiments, the antibody that specifically binds to human ILT2 comprises a heavy chain comprising an amino acid sequence which is at least 90%, 95%, 96%, 97%, 98%, 99%, or 100% identical to the amino acid sequence set forth in SEQ ID NO: 9; and a light chain comprising an amino acid sequence which is at least 90%, 95%, 96%, 97%, 98%, 99%, or 100% identical to the amino acid sequence set forth in SEQ ID NO: 10. In certain embodiments, the antibody comprises a heavy chain comprising the amino acid sequence set forth in SEQ ID NO: 9 and a light chain comprising the amino acid sequence set forth in SEQ ID NO: 10. In certain embodiments, the amino acid sequence of the heavy chain consists of the amino acid sequence set forth in SEQ ID NO: 9 and the amino acid sequence of the light chain consists of the amino acid sequence set forth in SEQ ID NO: 10.

In certain embodiments the antibody that specifically binds to human ILT2 is AGEN1571.

The amino acid sequences of exemplary anti-ILT2 antibodies are provided in Table 1 herein.

TABLE 1
Amino acid sequences of exemplary anti-ILT2 antibodies.

| Description | SEQ ID NO: | Amino acid sequence |
|---|---|---|
| CDRH1 | 1 | RYYMH |
| CDRH2 | 2 | WINPYTGGTNYAQKFQG |
| CDRH3 | 3 | KQPSSDY |
| CDRL1 | 4 | RSSQSLLHSNTYNYLD |
| CDRL2 | 5 | LGSNRAS |
| CDRL3 | 6 | LQASQAPWT |
| VH | 7 | QVQLVQSGAEVKKPGASVKVSCKASGYTFNRYYMHW |
| | | VRQAPGQGLEWMGWINPYTGGTNYAQKFQGRVTMTR |
| | | DTSISTAYMELSRLRSDDTAVYYCASKQPSSDYWGQGT |
| | | LVTVSS |
| VL | 8 | DIVMTQSPLSLPVTPGEPASISCRSSQSLLHSNTYNYLD |
| | | WYLQKPGQSPQLLIYLGSNRASGVPDRFSGSGSGTDFTL |
| | | KISRVEAEDVGVYYCLQASQAPWTFGQGTKVEIK |
| Heavy chain | 9 | QVQLVQSGAEVKKPGASVKVSCKASGYTFNRYYMHW |
| | | VRQAPGQGLEWMGWINPYTGGTNYAQKFQGRVTMTR |
| | | DTSISTAYMELSRLRSDDTAVYYCASKQPSSDYWGQGT |
| | | LVTVSSASTKGPSVFPLAPCSRSTSESTAALGCLVKDYF |
| | | PEPVTVSWNSGALTSGVHTFPAVLQSSGLYSLSSVVTVP |
| | | SSSLGTKTYTCNVDHKPSNTKVDKRVESKYGPPCPPCP |
| | | APEFLGGPSVFLFPPKPKDTLMISRTPEVTCVVVDVSQE |
| | | DPEVQFNWYVDGVEVHNAKTKPREEQFNSTYRVVSVL |
| | | TVLHQDWLNGKEYKCKVSNKGLPSSIEKTISKAKGQPR |
| | | EPQVYTLPPSQEEMTKNQVSLTCLVKGFYPSDIAVEWE |
| | | SNGQPENNYKTTPPVLDSDGSFFLYSRLTVDKSRWQEG |
| | | NVFSCSVMHEALHNHYTQKSLSLSLG |
| Light chain | 10 | DIVMTQSPLSLPVTPGEPASISCRSSQSLLHSNTYNYLD |
| | | WYLQKPGQSPQLLIYLGSNRASGVPDRFSGSGSGTDFTL |
| | | KISRVEAEDVGVYYCLQASQAPWTFGQGTKVEIKRTVA |
| | | APSVFIFPPSDEQLKSGTASVVCLLNNFYPREAKVQWK |
| | | VDNALQSGNSQESVTEQDSKDSTYSLSSTLTLSKADYE |
| | | KHKVYACEVTHQGLSSPVTKSFNRGEC |

### PD-1 Inhibitors

Human PD-1 inhibitors that are useful in the methods and uses described herein include but are not limited to those described below.

In certain embodiments, the human PD-1 inhibitor is an antibody that specifically binds to human PD-1 or human PD-L1. In certain embodiments, the antibody that specifically binds to human PD-1 comprises: a heavy chain variable region (VH) comprising the CDRH1, CDRH2, and CDRH3 amino acid sequences of the VH amino acid sequence set forth in SEQ ID NO: 17. In certain embodiments, the antibody that specifically binds to human PD-1 comprises: a light chain variable region (VL) comprising the CDRL1, CDRL2, and CDRL3 amino acid sequences of the VL amino acid sequence set forth in SEQ ID NO: 18. In certain embodiments, the antibody that specifically binds to human PD-1 comprises a heavy chain variable region (VH) comprising the CDRH1, CDRH2, and CDRH3 amino acid sequences of the VH amino acid sequence set forth in SEQ ID NO: 17 and a light chain variable region (VL) comprising the CDRL1, CDRL2, and CDRL3 amino acid sequences of the VL amino acid sequence set forth in SEQ ID NO: 18.

In certain embodiments, the antibody that specifically binds to human PD-1 comprises the CDRH1, CDRH2, and CDRH3 amino acid sequences set forth in SEQ ID NO: 11, 12, and 13, respectively. In certain embodiments, the antibody that specifically binds to human PD-1 comprises the CDRL1, CDRL2, and CDRL3 amino acid sequences set forth in SEQ ID NO: 14, 15, and 16, respectively. In certain embodiments, the antibody that specifically binds to human PD-1 comprises the CDRH1, CDRH2, CDRH3, CDRL1, CDRL2, and CDRL3 amino acid sequences set forth in SEQ ID NOs: 11, 12, 13, 14, 15, and 16, respectively.

In certain embodiments, the antibody that specifically binds to human PD-1 comprises a VH comprising an amino acid sequence which is at least 90%, 95%, 96%, 97%, 98%, 99%, or 100% identical to the amino acid sequence set forth in SEQ ID NO: 17. In certain embodiments, the antibody that specifically binds to human PD-1 comprises a VH comprising the amino acid sequence set forth in SEQ ID NO: 17.

In certain embodiments, the antibody that specifically binds to human PD-1 comprises a VL comprising an amino acid sequence which is at least 90%, 95%, 96%, 97%, 98%, 99%, or 100% identical to the amino acid sequence set forth in SEQ ID NO: 18. In certain embodiments, the antibody that specifically binds to human PD-1 comprises a VL comprising the amino acid sequence set forth in SEQ ID NO: 18.

In certain embodiments, the antibody that specifically binds to human PD-1 comprises: a VH comprising an amino acid sequence which is at least 90%, 95%, 96%, 97%, 98%, 99%, or 100% identical to the amino acid sequence set forth in SEQ ID NO: 17; and a VL comprising an amino acid sequence which is at least 90%, 95%, 96%, 97%, 98%, 99%, or 100% identical to the amino acid sequence set forth in SEQ ID NO: 18. In certain embodiments, the antibody that specifically binds to human PD-1 comprises a VH comprising the amino acid sequence set forth in SEQ ID NO: 17; and a VL comprising the amino acid sequence set forth in SEQ ID NO: 18.

In certain embodiments, the antibody that specifically binds to human PD-1 comprises a heavy chain constant region selected from the group consisting of human IgG₁, IgG₂, IgG₃, IgG₄, IgA₁, and IgA₂. In certain embodiments, the heavy chain constant region is IgG₁. In certain embodiments, the heavy chain constant region is IgG₂. In certain embodiments, the antibody comprises a light chain constant region selected from the group consisting of a human kappa light chain constant region and a human lambda light chain constant region.

In certain embodiments, the antibody that specifically binds to human PD-1 comprises an IgG₄ heavy chain constant region. In certain embodiments, the heavy chain constant region is a human IgG₄ heavy chain constant region comprising a P at position 228, numbered according to the EU numbering system.

In certain embodiments, the antibody that specifically binds to human PD-1 comprises a heavy chain comprising an amino acid sequence which is at least 90%, 95%, 96%, 97%, 98%, 99%, or 100% identical to the amino acid sequence set forth in SEQ ID NO: 19. In certain embodiments, the antibody comprises a heavy chain comprising the amino acid sequence set forth in SEQ ID NO: 19.

In certain embodiments, the antibody that specifically binds to human PD-1 comprises a light chain comprising an amino acid sequence which is at least 90%, 95%, 96%, 97%, 98%, 99%, or 100% identical to the amino acid sequence set forth in SEQ ID NO: 20. In certain embodiments, the antibody comprises a light chain comprising the amino acid sequence set forth in SEQ ID NO: 20.

In certain embodiments, the antibody that specifically binds to human PD-1 comprises a heavy chain comprising an amino acid sequence which is at least 90%, 95%, 96%, 97%, 98%, 99%, or 100% identical to the amino acid sequence set forth in SEQ ID NO: 19; and a light chain comprising an amino acid sequence which is at least 90%, 95%, 96%, 97%, 98%, 99%, or 100% identical to the amino acid sequence set forth in SEQ ID NO: 20. In certain embodiments, the antibody that specifically binds to human PD-1 comprises a heavy chain comprising the amino acid sequence set forth in SEQ ID NO: 19 and a light chain comprising the amino acid sequence set forth in SEQ ID NO: 20. In certain embodiments, the amino acid sequence of the heavy chain consists of the amino acid sequence set forth in SEQ ID NO: 19 and the amino acid sequence of the light chain consists of the amino acid sequence set forth in SEQ ID NO: 20.

In certain embodiments the antibody that specifically binds to human PD-1 is balstilimab.

The amino acid sequences of exemplary anti-PD-1 antibodies are provided in Table 2 herein.

TABLE 2
Amino acid sequences of exemplary anti-PD-1 antibodies.

| Description | SEQ ID NO: | Amino acid sequence |
|---|---|---|
| CDRH1 | 11 | SYGMH |
| CDRH2 | 12 | VIWYDGSNKYYADSVKG |
| CDRH3 | 13 | NGDH |
| CDRL1 | 14 | RASQSVSSNLA |
| CDRL2 | 15 | GASTRAT |
| CDRL3 | 16 | QQYNNWPRT |
| VH | 17 | QVQLVESGGGVVQPGRSLRLSCAASGFTFSSYGMHWV |
| | | RQAPGKGLEWVAVIWYDGSNKYYADSVKGRFTISRD |
| | | NSKNTLYLQMNSLRAEDTAVYYCASNGDHWGQGTLV |
| | | TVSS |
| VL | 18 | EIVMTQSPATLSVSPGERATLSCRASQSVSSNLAWYQQ |
| | | KPGQAPRLLIYGASTRATGIPARFSGSGSGTEFTLTISSL |
| | | QSEDFAVYYCQQYNNWPRTFGQGTKVEIK |
| Heavy chain | 19 | QVQLVESGGGVVQPGRSLRLSCAASGFTFSSYGMHWV |
| | | RQAPGKGLEWVAVIWYDGSNKYYADSVKGRFTISRD |
| | | NSKNTLYLQMNSLRAEDTAVYYCASNGDHWGQGTLV |
| | | TVSSASTKGPSVFPLAPCSRSTSESTAALGCLVKDYFPE |
| | | PVTVSWNSGALTSGVHTFPAVLQSSGLYSLSSVVTVPS |
| | | SSLGTKTYTCNVDHKPSNTKVDKRVESKYGPPCPPCPA |
| | | PEFLGGPSVFLFPPKPKDTLMISRTPEVTCVVVDVSQED |
| | | PEVQFNWYVDGVEVHNAKTKPREEQFNSTYRVVSVLT |
| | | VLHQDWLNGKEYKCKVSNKGLPSSIEKTISKAKGQPR |
| | | EPQVYTLPPSQEEMTKNQVSLTCLVKGFYPSDIAVEWE |
| | | SNGQPENNYKTTPPVLDSDGSFFLYSRLTVDKSRWQE |
| | | GNVFSCSVMHEALHNHYTQKSLSLSLG |
| Light chain | 20 | EIVMTQSPATLSVSPGERATLSCRASQSVSSNLAWYQQ |
| | | KPGQAPRLLIYGASTRATGIPARFSGSGSGTEFTLTISSL |
| | | QSEDFAVYYCQQYNNWPRTFGQGTKVEIKRTVAAPSV |
| | | FIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |

In certain embodiments the antibody that specifically binds to human PD-1 or human PD-L1 is adebrelimab, atezolizumab, avelumab, camrelizumab, cemiplimab, cosibelimab, dostarlimab, durvalumab, enlonstobart, envafolimab, nivolumab, pembrolizumab, penpulimab, pidilizumab, prolgolimab, pucotenlimab, retifanlimab, serplulimab, sintilimab, socazolimab, sugemalimab, tagitanlimab, tislelizumab, toripalimab, and zimberelimab.

Further non-limiting examples of anti-PD-1 antibodies that may be used in treatment methods described herein are disclosed in the following patents and patent applications, which are incorporated herein by reference in their entireties for all purposes: U.S. Pat. Nos. 6,808,710; 7,332,582; 7,488,802; 8,008,449; 8,114,845; 8,168,757; 8,354,509; 8,686,119; 8,735,553; 8,747,847; 8,779,105; 8,927,697; 8,993,731; 9,102,727; 9,205,148; U.S. Publication No. US 2013/0202623 A1; U.S. Publication No. US 2013/0291136 A1; U.S. Publication No. US 2014/0044738 A1; U.S. Publication No. US 2014/0356363 A1; U.S. Publication No. US 2016/0075783 A1; and PCT Publication No. WO 2013/033091 A1; PCT Publication No. WO 2015/036394 A1; PCT Publication No. WO 2014/179664 A2; PCT Publication No. WO 2014/209804 A1; PCT Publication No. WO 2014/206107 A1; PCT Publication No. WO 2015/058573 A1; PCT Publication No. WO 2015/085847 A1; PCT Publication No. WO 2015/200119 A1; PCT Publication No. WO 2016/015685 A1; and PCT Publication No. WO 2016/020856 A1.

Further examples of anti-PD-L1 antibodies that may be used in treatment methods described herein are disclosed in the following patents and patent applications, which are incorporated herein by reference in their entireties for all purposes: U.S. Pat. Nos. 7,943,743; 8,168,179; 8,217,149; 8,552,154; 8,779,108; 8,981,063; 9,175,082; U.S. Publication No. US 2010/0203056 A1; U.S. Publication No. US 2003/0232323 A1; U.S. Publication No. US 2013/0323249 A1; U.S. Publication No. US 2014/0341917 A1; U.S. Publication No. US 2014/0044738 A1; U.S. Publication No. US 2015/0203580 A1; U.S. Publication No. US 2015/0225483 A1; U.S. Publication No. US 2015/0346208 A1; U.S. Publication No. US 2015/0355184 A1; and PCT Publication No. WO 2014/100079 A1; PCT Publication No. WO 2014/022758 A1; PCT Publication No. WO 2014/055897 A2; PCT Publication No. WO 2015/061668 A1; PCT Publication No. WO 2015/109124 A1; PCT Publication No. WO 2015/195163 A1; PCT Publication No. WO 2016/000619 A1; and PCT Publication No. WO 2016/030350 A1.

In certain embodiments, the human PD-1 inhibitor is pidilizumab.

### CTLA-4 Inhibitors

Human CTLA-4 inhibitors that are useful in the methods and uses described herein include, but are not limited to, those described below.

In certain embodiments, the human CTLA-4 inhibitor is an antibody that specifically binds to human CTLA-4. In certain embodiments, the antibody that specifically binds to human CTLA-4 comprises: a heavy chain variable region (VH) comprising the CDRH1, CDRH2, and CDRH3 amino acid sequences of the VH amino acid sequence set forth in SEQ ID NO: 27. In certain embodiments, the antibody that specifically binds to human CTLA-4 comprises: a light chain variable region (VL) comprising the CDRL1, CDRL2, and CDRL3 amino acid sequences of the VL amino acid sequence set forth in SEQ ID NO: 28. In certain embodiments, the antibody comprises a heavy chain variable region (VH) comprising the CDRH1, CDRH2, and CDRH3 amino acid sequences of the VH amino acid sequence set forth in SEQ ID NO: 27 and a light chain variable region (VL) comprising the CDRL1, CDRL2, and CDRL3 amino acid sequences of the VL amino acid sequence set forth in SEQ ID NO: 28.

In certain embodiments, the antibody that specifically binds to human CTLA-4 comprises the CDRH1, CDRH2, and CDRH3 amino acid sequences set forth in SEQ ID NOs: 21, 22, and 23, respectively. In certain embodiments, the antibody that specifically binds to human CTLA-4 comprises the CDRL1, CDRL2, and CDRL3 amino acid sequences set forth in SEQ ID NOs: 24, 25, and 26, respectively. In certain embodiments, the antibody comprises the CDRH1, CDRH2, CDRH3, CDRL1, CDRL2, and CDRL3 amino acid sequences set forth in SEQ ID NOs: 21, 22, 23, 24, 25, and 26, respectively.

In certain embodiments, the antibody that specifically binds to human CTLA-4 comprises a VH comprising an amino acid sequence which is at least 90%, 95%, 96%, 97%, 98%, 99%, or 100% identical to the amino acid sequence set forth in SEQ ID NO: 27. In certain embodiments, the antibody comprises a VH comprising the amino acid sequence set forth in SEQ ID NO: 27.

In certain embodiments, the antibody that specifically binds to human CTLA-4 comprises a VL comprising an amino acid sequence which is at least 90%, 95%, 96%, 97%, 98%, 99%, or 100% identical to the amino acid sequence set forth in SEQ ID NO: 28. In certain embodiments, the antibody comprises a VL comprising the amino acid sequence set forth in SEQ ID NO: 28.

In certain embodiments, the antibody that specifically binds to human CTLA-4 comprises: a VH comprising an amino acid sequence which is at least 90%, 95%, 96%, 97%, 98%, 99%, or 100% identical to the amino acid sequence set forth in SEQ ID NO: 27; and a VL comprising an amino acid sequence which is at least 90%, 95%, 96%, 97%, 98%, 99%, or 100% identical to the amino acid sequence set forth in SEQ ID NO: 28. In certain embodiments, the antibody comprises a VH comprising the amino acid sequence set forth in SEQ ID NO: 27 and a VL comprising the amino acid sequence set forth in SEQ ID NO: 28.

In certain embodiments, the antibody that specifically binds to human CTLA-4 comprises a heavy chain constant region selected from the group consisting of human IgG₁, IgG₂, IgG₃, IgG₄, IgA₁, and IgA₂. In certain embodiments, the heavy chain constant region is IgG₁. In certain embodiments, the heavy chain constant region is IgG₂. In certain embodiments, the antibody that specifically binds to human CTLA-4 comprises an IgG₁ heavy chain constant region.

In certain embodiments, the antibody comprises a light chain constant region selected from the group consisting of a human kappa light chain constant region and a human lambda light chain constant region.

In certain embodiments, the antibody that specifically binds to human CTLA-4 comprises a human IgG heavy chain constant region that is a variant of a wild-type human IgG heavy chain constant region, wherein the variant human IgG heavy chain constant region binds to FcγRIIIA with a higher affinity than the wild-type human IgG heavy chain constant region binds to FcγRIIIA.

In certain embodiments, the IgG region of the antibody that specifically binds to human CTLA-4 has an increased affinity for FcγRIIIA, e.g., as compared with an antibody with a wild-type Fc region, e.g., an IgG₁ Fc. Sequence alterations that result in increased affinity for FcγRIIIA are known in the art, for example, in Kellner et al., Methods 65:105-113 (2014), Lazar et al., Proc Natl Acad Sci 103:4005-4010 (2006), Shields et al., J Biol Chem. 276(9):6591-6604 (2001), each of which is herein incorporated by reference in its entirety.

In certain embodiments, the antibody that specifically binds to human CTLA-4 is afucosylated.

In certain embodiments, the antibody that specifically binds to human CTLA-4 comprises a heavy chain constant region, e.g., an IgG₁ constant region, or fragment thereof comprising a mutation selected from the group consisting of: L235V, G236A, S239D, F243L, T256A, K290A, R292P, S298A, Y300L, V305I, A330L, I332E, E333A, K334A, A339T, and P396L, and combinations thereof, numbered according to the EU numbering system.

In certain embodiments, the antibody that specifically binds to human CTLA-4 comprises a heavy chain constant region, e.g., an IgG₁ constant region, or fragment thereof comprising S239D, numbered according to the EU numbering system. In certain embodiments, the antibody that specifically binds to human CTLA-4 comprises a heavy chain constant region, e.g., an IgG₁ constant region, or fragment thereof comprising T256A, numbered according to the EU numbering system. In certain embodiments, the antibody that specifically binds to human CTLA-4 comprises a heavy chain constant region, e.g., an IgG₁ constant region, or fragment thereof comprising K290A, numbered according to the EU numbering system. In certain embodiments, the antibody that specifically binds to human CTLA-4 comprises a heavy chain constant region, e.g., an IgG₁ constant region, or fragment thereof comprising S298A, numbered according to the EU numbering system. In certain embodiments, the antibody that specifically binds to human CTLA-4 comprises a heavy chain constant region, e.g., an IgG₁ constant region, or fragment thereof comprising I332E, numbered according to the EU numbering system. In certain embodiments, the antibody that specifically binds to human CTLA-4 comprises a heavy chain constant region, e.g., an IgG₁ constant region, or fragment thereof comprising E333A, numbered according to the EU numbering system. In certain embodiments, the antibody that specifically binds to human CTLA-4 comprises a heavy chain constant region, e.g., an IgG₁ constant region, or fragment thereof comprising K334A, numbered according to the EU numbering system. In certain embodiments, the antibody that specifically binds to human CTLA-4 comprises a heavy chain constant region, e.g., an IgG₁ constant region, or fragment thereof comprising A339T, numbered according to the EU numbering system. In certain embodiments, the antibody that specifically binds to human CTLA-4 comprises a heavy chain constant region, e.g., an IgG₁ constant region, or fragment thereof comprising S239D and I332E, numbered according to the EU numbering system. In certain embodiments, the antibody that specifically binds to human CTLA-4 comprises an IgG₁ heavy chain constant region that comprises S239D/I332E mutations, numbered according to the EU numbering system. In certain embodiments, the antibody that specifically binds to human CTLA-4 comprises a heavy chain constant region, e.g., an IgG₁ constant region, or fragment thereof comprising S239D, A330L, and I332E, numbered according to the EU numbering system. In certain embodiments, the antibody that specifically binds to human CTLA-4 comprises an IgG₁ heavy chain constant region that comprises S239D/A330L/I332E mutations, numbered according to the EU numbering system. In certain embodiments, the antibody that specifically binds to human CTLA-4 comprises a heavy chain constant region, e.g., an IgG₁ constant region, or fragment thereof comprising S298A, E333A, and K334A, numbered according to the EU numbering system. In certain embodiments, the antibody that specifically binds to human CTLA-4 comprises a heavy chain constant region, e.g., an IgG₁ constant region, or fragment thereof comprising G236A, S239D, and I332E, numbered according to the EU numbering system. In certain embodiments, the antibody that specifically binds to human CTLA-4 comprises a heavy chain constant region, e.g., an IgG₁ constant region, or fragment thereof comprising F243L, R292P, Y300L, V305I, and P396L, numbered according to the EU numbering system. In certain embodiments, the antibody that specifically binds to human CTLA-4 comprises an IgG₁ heavy chain constant region that comprises L235V/F243L/R292P/Y300L/P396L mutations, numbered according to the EU numbering system. In certain embodiments, the heavy chain constant region is a human IgG₁ heavy chain constant region comprising a D at position 239, a L at position 330, and an E at position 332, numbered according to the EU numbering system.

In certain embodiments, the antibody that specifically binds to human CTLA-4 comprises a heavy chain comprising an amino acid sequence which is at least 90%, 95%, 96%, 97%, 98%, 99%, or 100% identical to the amino acid sequence set forth in SEQ ID NO: 29. In certain embodiments, the antibody comprises a heavy chain comprising the amino acid sequence set forth in SEQ ID NO: 29.

In certain embodiments, the antibody that specifically binds to human CTLA-4 comprises a light chain comprising an amino acid sequence which is at least 90%, 95%, 96%, 97%, 98%, 99%, or 100% identical to the amino acid sequence set forth in SEQ ID NO: 30. In certain embodiments, the antibody comprises a light chain comprising the amino acid sequence set forth in SEQ ID NO: 30.

In certain embodiments, the antibody that specifically binds to human CTLA-4 comprises a heavy chain comprising an amino acid sequence which is at least 90%, 95%, 96%, 97%, 98%, 99%, or 100% identical to the amino acid sequence set forth in SEQ ID NO: 29; and a light chain comprising an amino acid sequence which is at least 90%, 95%, 96%, 97%, 98%, 99%, or 100% identical to the amino acid sequence set forth in SEQ ID NO: 30. In certain embodiments, the antibody comprises a heavy chain comprising the amino acid sequence set forth in SEQ ID NO: 29 and a light chain comprising the amino acid sequence set forth in SEQ ID NO: 30. In certain embodiments, the amino acid sequence of the heavy chain consists of the amino acid sequence set forth in SEQ ID NO: 29 and the amino acid sequence of the light chain consists of the amino acid sequence set forth in SEQ ID NO: 30.

In certain embodiments the antibody that specifically binds to human CTLA-4 is selected from the group consisting of botensilimab, ipilimumab, and tremelimumab. In certain embodiments the antibody that specifically binds to human CTLA-4 is botensilimab.

The amino acid sequences of exemplary anti-CTLA-4 antibodies are provided in Table 3 herein.

TABLE 3
Amino acid sequences of exemplary anti-CTLA-4 antibodies.

| Description | SEQ ID NO: | Amino acid sequence |
|---|---|---|
| CDRH1 | 21 | SYSMN |
| CDRH2 | 22 | SISSSSSYIYYAESVKG |
| CDRH3 | 23 | VGLFGPFDI |
| CDRL1 | 24 | RASQSVSRYLG |
| CDRL2 | 25 | GASTRAT |
| CDRL3 | 26 | QQYGSSPWT |
| VH | 27 | EVQLVESGGGLVKPGGSLRLSCAASGFTFSSYSMNWVR |
| | | QAPGKGLEWVSSISSSSSYIYYAESVKGRFTISRDNAKN |
| | | SLYLQMNSLRAEDTAVYYCARVGLFGPFDIWGQGTLV |
| | | TVSS |
| VL | 28 | EIVLTQSPGTLSLSPGERATLSCRASQSVSRYLGWYQQK |
| | | PGQAPRLLIYGASTRATGIPDRFSGSGSGTDFTLTITRLEP |
| | | EDFAVYYCQQYGSSPWTFGQGTKVEIK |
| Heavy chain | 29 | EVQLVESGGGLVKPGGSLRLSCAASGFTFSSYSMNWVR |
| | | QAPGKGLEWVSSISSSSSYIYYAESVKGRFTISRDNAKN |
| | | SLYLQMNSLRAEDTAVYYCARVGLFGPFDIWGQGTLV |
| | | TVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPC |
| | | PAPELLGGPDVFLFPPKPKDTLMISRTPEVTCVVVDVSH |
| | | EDPEVKFNWYVDGVEVHNAKTKPREEQYNSTYRVVSV |
| | | LTVLHQDWLNGKEYKCKVSNKALPLPEEKTISKAKGQP |
| | | REPQVYTLPPSREEMTKNQVSLTCLVKGFYPSDIAVEW |
| | | ESNGQPENNYKTTPPVLDSDGSFFLYSKLTVDKSRWQQ |
| | | GNVFSCSVMHEALHNHYTQKSLSLSPG |
| Light chain | 30 | EIVLTQSPGTLSLSPGERATLSCRASQSVSRYLGWYQQK |
| | | PGQAPRLLIYGASTRATGIPDRFSGSGSGTDFTLTITRLEP |
| | | EDFAVYYCQQYGSSPWTFGQGTKVEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYAC |
| | | EVTHQGLSSPVTKSFNRGEC |

### Methods of Treating Cancer

The present disclosure provides methods for the treatment of cancer using an antibody that specifically binds to human ILT2, and combinations of the antibody with a human CTLA-4 inhibitor (e.g., an antibody that specifically binds to human CTLA-4) and a human PD-1 inhibitor (e.g., an antibody that specifically binds to human PD-1).

The present disclosure also provides methods for the treatment of cancer using an antibody that specifically binds to human ILT2, and combinations of the antibody with a checkpoint targeting agent (e.g., a CTLA-4 inhibitor, a PD-L1 inhibitor, a PD-L2 inhibitor, or an PD-1 inhibitor). In some embodiments, a checkpoint targeting agent comprises an antagonist anti-CTLA-4 antibody, an antagonist anti-PD-L1 antibody, an antagonist anti-PD-L2 antibody, or an antagonist anti-PD-1 antibody. In some embodiments, a checkpoint targeting agent comprises a small molecule (e.g., inhibitory) targeting CTLA-4, PD-L1 antibody, PD-L2, or PD-1. In some embodiments, a checkpoint targeting agent comprises a peptide (e.g., inhibitory) targeting CTLA-4, PD-L1 antibody, PD-L2, or PD-1. In some embodiments, a checkpoint targeting agent comprises a Fc fusion protein (e.g., inhibitory IgG-Fc tagged protein) targeting CTLA-4, PD-L1 antibody, PD-L2, or PD-1.

The present disclosure also provides methods of enhancing an immune response in a subject in need thereof using an antibody that specifically binds to human ILT2, and combinations of the antibody with a checkpoint targeting agent (e.g., a CTLA-4 inhibitor, a PD-L1 inhibitor, a PD-L2 inhibitor, or an PD-1 inhibitor). In certain embodiments, methods disclosed herein enhance an immune response in a subject in need thereof, the method comprises using an antibody that specifically binds to human ILT2, and combinations of the antibody with a human CTLA-4 inhibitor (e.g., an antibody that specifically binds to human CTLA-4) and/or a human PD-1 inhibitor (e.g., an antibody that specifically binds to human PD-1). In some embodiments, the enhanced immune response comprises increased CD25 expression on a T cell, an NK cell, and/or an NKT cell as compared to a reference or control. In some embodiments, the enhanced immune response comprises increased TNF alpha, IFN gamma, and/or IL-1 beta cytokine secretion as compared to a reference or control. In some embodiments, the T cell is a CD8⁺ T cell. In some embodiments, the NK cell is CD3/CD56⁺ NK cell In some embodiments, the NKT cell is CD3⁺/CD56⁺ NKT cell. In some embodiments, the enhanced immune response comprises enhancing FcγR-mediated immune activation as compared to a reference or control. In some embodiments, the FcγR-mediated immune activation comprises FcγRIIIA receptor (CD16) signaling. In some embodiments, the reference or control is a published or historical reference of an immune response in a subject to an ILT2 inhibitor. In some embodiments, the reference or control is a published or historical reference of an immune response in a subject to a CTLA-4 inhibitor. In some embodiments, the reference or control is a published or historical reference of an immune response in a subject to a PD-1 inhibitor.

In one aspect, the present disclosure provides a method of treating cancer in a subject in need thereof, the method comprising administering to the subject a first antibody that specifically binds to human ILT2 at a dose of about 1 mg to about 2000 mg, wherein the first antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

In another aspect, provided herein is a method of treating cancer in a subject in need thereof, the method comprising administering to the subject: a first antibody that specifically binds to human ILT2, wherein the antibody comprises: a VH comprising the CDRH1, CDRH2, and CDRH3 amino acid sequences of the VH amino acid sequence set forth in SEQ ID NO: 7; and a VL comprising the CDRL1, CDRL2, and CDRL3 amino acid sequences of the VL amino acid sequence set forth in SEQ ID NO: 8; and a human PD-1 inhibitor.

In another aspect, provided herein is a method of treating cancer in a subject in need thereof, the method comprising administering to the subject: a first antibody that specifically binds to human ILT2, wherein the antibody comprises: a VH comprising the CDRH1, CDRH2, and CDRH3 amino acid sequences of the VH amino acid sequence set forth in SEQ ID NO: 7; and a VL comprising the CDRL1, CDRL2, and CDRL3 amino acid sequences of the VL amino acid sequence set forth in SEQ ID NO: 8; and a human CTLA-4 inhibitor.

In another aspect, provided herein is a method of treating cancer in a subject in need thereof, the method comprising administering to the subject: a first antibody that specifically binds to human ILT2, wherein the antibody comprises: a VH comprising the CDRH1, CDRH2, and CDRH3 amino acid sequences of the VH amino acid sequence set forth in SEQ ID NO: 7; and a VL comprising the CDRL1, CDRL2, and CDRL3 amino acid sequences of the VL amino acid sequence set forth in SEQ ID NO: 8; a human PD-1 inhibitor; and a human CTLA-4 inhibitor.

The methods described herein can comprise a first antibody that specifically binds to human ILT2, given as a monotherapy. In certain embodiments, the methods described herein comprise any combination of a first antibody that specifically binds to human ILT2 described herein, and a human PD-1 inhibitor described herein and/or a human CTLA-4 inhibitor described herein.

In certain embodiments, the method comprises administration of a first antibody that specifically binds to human ILT2, and a human PD-1 inhibitor that is a second antibody that specifically binds to human PD-1 or human PD-L1. In certain embodiments, the second antibody specifically binds to human PD-1. In certain embodiments, the method comprises administration of a first antibody that specifically binds to human ILT2, and a second antibody comprising: a VH comprising the CDRH1, CDRH2, and CDRH3 amino acid sequences of the VH amino acid sequence set forth in SEQ ID NO: 17; and a VL comprising the CDRL1, CDRL2, and CDRL3 amino acid sequences of the VL amino acid sequence set forth in SEQ ID NO: 18. In certain embodiments, the second antibody comprises the CDRH1, CDRH2, CDRH3, CDRL1, CDRL2, and CDRL3 amino acid sequences, set forth in SEQ ID NOs: 11, 12, 13, 14, 15, and 16, respectively. In certain embodiments, the second antibody comprises the VH amino acid sequence of SEQ ID NO: 17, and/or the VL amino acid sequence of SEQ ID NO: 18. In certain embodiments, the second antibody comprises the VH and VL amino acid sequences of SEQ ID NOs: 17 and 18, respectively. In certain embodiments, the second antibody comprises a heavy chain constant region selected from the group consisting of human IgG₁, IgG₂, IgG₃, IgG₄, IgA₁, IgA₂, and IgM. In certain embodiments, the second antibody comprises a heavy chain constant region that is a human IgG₄ heavy chain constant region comprising a P at position 228, numbered according to the EU numbering system. In certain embodiments, the second antibody comprises a heavy chain comprising the amino acid sequence of SEQ ID NO: 19, and/or a light chain comprising the amino acid sequence of SEQ ID NO: 20. In certain embodiments, the second antibody comprises a heavy chain and a light chain comprising the amino acid sequences of SEQ ID NOs: 19 and 20, respectively. In certain embodiments, the second antibody is selected from the group consisting of balstilimab, adebrelimab, atezolizumab, avelumab, camrelizumab, cemiplimab, cosibelimab, dostarlimab, durvalumab, enlonstobart, envafolimab, nivolumab, pembrolizumab, penpulimab, pidilizumab, prolgolimab, pucotenlimab, retifanlimab, serplulimab, sintilimab, socazolimab, sugemalimab, tagitanlimab, tislelizumab, toripalimab, and zimberelimab. In certain embodiments, the second antibody is balstilimab.

In certain embodiments, the method comprises administration of a first antibody that specifically binds to human ILT2, and a human CTLA-4 inhibitor that is a third antibody that specifically binds to human CTLA-4. In certain embodiments, the method comprises administration of a first antibody that specifically binds to human ILT2, and a third antibody comprising: a VH comprising the CDRH1, CDRH2, and CDRH3 amino acid sequences of the VH amino acid sequence set forth in SEQ ID NO: 27; and a VL comprising the CDRL1, CDRL2, and CDRL3 amino acid sequences of the VL amino acid sequence set forth in SEQ ID NO: 28. In certain embodiments, the third antibody comprises the CDRH1, CDRH2, CDRH3, CDRL1, CDRL2, and CDRL3 amino acid sequences, set forth in SEQ ID NOs: 21, 22, 23, 24, 25, and 26, respectively. In certain embodiments, the third antibody comprises the VH amino acid sequence of SEQ ID NO: 27, and/or the VL amino acid sequence of SEQ ID NO: 28. In certain embodiments, the third antibody comprises the VH and VL amino acid sequences of SEQ ID NOs: 27 and 28, respectively. In certain embodiments, the third antibody comprises a heavy chain constant region selected from the group consisting of human IgG₁, IgG₂, IgG₃, IgG₄, IgA₁, IgA₂, and IgM. In certain embodiments, the third antibody comprises a heavy chain constant region that is a human IgG₁ heavy chain constant region comprising a D at position 239, a L at position 330, and an E at position 332, numbered according to the EU numbering system. In certain embodiments, the third antibody comprises a heavy chain comprising the amino acid sequence of SEQ ID NO: 29, and/or a light chain comprising the amino acid sequence of SEQ ID NO: 30. In certain embodiments, the third antibody comprises a heavy chain and a light chain comprising the amino acid sequences of SEQ ID NOs: 29 and 30, respectively. In certain embodiments, the third antibody is selected from the group consisting of botensilimab, ipilimumab, and tremelimumab. In certain embodiments, the third antibody is botensilimab.

In certain embodiments, the method comprises administration of a first antibody that specifically binds to human ILT2, a second antibody that binds specifically binds to human PD-1, and a third antibody that specifically binds to human CTLA-4.

In certain embodiments, the first antibody is administered at a dose of about 1 mg to about 2000 mg, about 1 mg to about 1000 mg, about 3 mg to about 300 mg, about 10 mg to about 100 mg, about 1 mg to about 600 mg, about 1 mg to about 300 mg, about 1 mg to about 100 mg, about 1 mg to about 30 mg, about 1 mg to about 10 mg, about 1 mg to about 3 mg, about 10 mg to about 2000 mg, about 10 mg to about 1000 mg, about 10 mg to about 600 mg, about 10 mg to about 300 mg, about 10 mg to about 30 mg, about 30 mg to about 1000 mg, about 30 mg to about 600 mg, about 30 mg to about 300 mg, about 30 mg to about 100 mg, about 100 mg to about 1000 mg, about 100 mg to about 600 mg, about 100 mg to about 300 mg, about 300 mg to about 2000 mg, about 300 mg to about 1000 mg, or about 300 mg to about 600 mg.

In certain embodiments, the first antibody is administered at a dose of about 1 mg, about 3 mg, about 10 mg, about 30 mg, about 100 mg, about 300 mg, about 400, about 500, about 600, about 700. about 800, about 900, about 1000 mg, about 1250, about 1500, about 1750, or about 2000.

In certain embodiments, the first antibody is administered once every week. In certain embodiments, the first antibody is administered once every 2 weeks. In certain embodiments, the first antibody is administered once every 3 weeks. In certain embodiments, the first antibody is administered once every 4 weeks. In certain embodiments, the first antibody is administered once every 5 weeks. In certain embodiments, the first antibody is administered once every 6 weeks.

In certain embodiments, the first antibody is administered at a dose of about 1 mg once every 3 weeks. In certain embodiments, the first antibody is administered at a dose of about 3 mg once every 3 weeks. In certain embodiments, the first antibody is administered at a dose of about 10 mg once every 3 weeks. In certain embodiments, the first antibody is administered at a dose of about 30 mg once every 3 weeks. In certain embodiments, the first antibody is administered at a dose of about 100 mg once every 3 weeks. In certain embodiments, the first antibody is administered at a dose of about 300 mg once every 3 weeks. In certain embodiments, the first antibody is administered at a dose of about 1000 mg once every 3 weeks. In certain embodiments, the first antibody is administered at a dose of about 1 mg once every 6 weeks. In certain embodiments, the first antibody is administered at a dose of about 3 mg once every 6 weeks. In certain embodiments, the first antibody is administered at a dose of about 10 mg once every 6 weeks. In certain embodiments, the first antibody is administered at a dose of about 30 mg once every 6 weeks. In certain embodiments, the first antibody is administered at a dose of about 100 mg once every 6 weeks. In certain embodiments, the first antibody is administered at a dose of about 300 mg once every 6 weeks. In certain embodiments, the first antibody is administered at a dose of about 1000 mg once every 6 weeks.

In certain embodiments, the human PD-1 inhibitor is administered at a dose of about 1 mg to 1000 mg, 100 mg to 750 mg, 100 mg to 500 mg, 100 mg to 450 mg, 150 mg to 750 mg, 150 mg to 500 mg, 150 mg to 450 mg, 200 mg to 750 mg, 200 mg to 500 mg, 200 mg to 450 mg, 300 mg to 750 mg, 300 mg to 500 mg, 300 mg to 450 mg, 400 mg to 750 mg, 400 mg to 500 mg, 400 mg to 450 mg, 450 mg to 750 mg, or 450 mg to 500 mg.

In certain embodiments, the human PD-1 inhibitor is administered at a dose of about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 450 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, or about 740 mg. In certain embodiments, the human PD-1 inhibitor is administered at a dose of about 240 mg. In certain embodiments, the human PD-1 inhibitor is administered at a dose of about 450 mg.

In certain embodiments, the human PD-1 inhibitor is administered at about 0.01 mg/kg, about 0.03 mg/kg, about 0.1 mg/kg, about 0.3 mg/kg, about 1 mg/kg, about 3 mg/kg, about 6 mg/kg, or about 10 mg/kg.

In certain embodiments, the human PD-1 inhibitor is administered once every week. In certain embodiments, the human PD-1 inhibitor is administered once every 2 weeks. In certain embodiments, the human PD-1 inhibitor is administered once every 3 weeks. In certain embodiments, the human PD-1 inhibitor is administered once every 4 weeks. In certain embodiments, the human PD-1 inhibitor is administered once every 5 weeks. In certain embodiments, the human PD-1 inhibitor is administered once every 6 weeks. In certain embodiments, the human PD-1 inhibitor is administered at a dose of 450 mg once every 3 weeks. In certain embodiments, the human PD-1 inhibitor is administered at a dose of 3 mg/kg mg once every 3 weeks.

In certain embodiments, the human CTLA-4 inhibitor is administered at a dose of about 1 mg to 1000 mg, 10 mg to 750 mg, 10 mg to 500 mg, 10 mg to 250 mg, 10 mg to 200 mg, 10 mg to 150 mg, 10 mg to 100 mg, 10 mg to 75 mg, 10 mg to 50 mg, 10 mg to 25 mg, 25 mg to 750 mg, 25 mg to 500 mg, 25 mg to 250 mg, 25 mg to 150 mg, 25 mg to 100 mg, 25 mg to 75 mg, 25 mg to 50 mg, 50 mg to 750 mg, 50 mg to 500 mg, 50 mg to 250 mg, 50 mg to 150 mg, 50 mg to 75 mg, 75 mg to 250 mg, 75 mg to 150 mg, 75 mg to 100 mg, 100 mg to 250 mg, 100 mg to 150 mg, or 150 mg to 250 mg.

In certain embodiments, the human CTLA-4 inhibitor is administered at a dose of about 10 mg, about 25 mg, about 50 mg, about 75 mg, about 100 mg, about 150 mg, about 200 mg, or about 250 mg. In certain embodiments, the human CTLA-4 inhibitor is administered at a dose of about 150 mg. In certain embodiments, the human CTLA-4 inhibitor is administered at a dose of about 75 mg. In certain embodiments, the human CTLA-4 inhibitor is administered at a dose of about 50 mg.

In certain embodiments, the human CTLA-4 inhibitor is administered at about 0.01 mg/kg, about 0.02 mg/kg, about 0.04 mg/kg, about 0.2 mg/kg, about 0.4 mg/kg, about 1 mg/kg, about 2 mg/kg, about 4 mg/kg, or about 6 mg/kg.

In certain embodiments, the human CTLA-4 inhibitor is administered once every week. In certain embodiments, the human CTLA-4 inhibitor is administered once every 2 weeks. In certain embodiments, the human CTLA-4 inhibitor is administered once every 3 weeks. In certain embodiments, the human CTLA-4 inhibitor is administered once every 4 weeks. In certain embodiments, the human CTLA-4 inhibitor is administered once every 5 weeks. In certain embodiments, the human CTLA-4 inhibitor is administered once every 6 weeks. In certain embodiments, the human CTLA-4 inhibitor is administered at a dose of 150 mg once every 6 weeks. In certain embodiments, the human CTLA-4 inhibitor is administered at a dose of 2 mg/kg mg once every 6 weeks. In certain embodiments, the human CTLA-4 inhibitor is administered at a dose of 4 mg/kg mg once every 6 weeks.

In certain embodiments, the first antibody is administered intravenously. In certain embodiments, the first antibody is administered intratumorally. In certain embodiments, the first antibody is administered peritumorally. In certain embodiments, the human PD-1 inhibitor or the human CTLA-4 inhibitor is administered intravenously. In certain embodiments, the human PD-1 inhibitor or the human CTLA-4 inhibitor is administered intratumorally. In certain embodiments, the human PD-1 inhibitor or the human CTLA-4 inhibitor is administered peritumorally.

In certain embodiments, the first antibody and the human PD-1 inhibitor are administered intravenously. In certain embodiments, the first antibody and the human PD-1 inhibitor are administered intratumorally. In certain embodiments, the first antibody and the human PD-1 inhibitor are administered peritumorally. In certain embodiments, the first antibody and the human CTLA-4 inhibitor are administered intravenously. In certain embodiments, the first antibody and the human CTLA-4 inhibitor are administered intratumorally. In certain embodiments, the first antibody and the human CTLA-4 inhibitor are administered peritumorally. In certain embodiments, the first antibody, the human PD-1 inhibitor, and the human CTLA-4 inhibitor are administered intravenously. In certain embodiments, the first antibody, the human PD-1 inhibitor, and the human CTLA-4 inhibitor are administered intratumorally. In certain embodiments, the first antibody, the human PD-1 inhibitor, and the human CTLA-4 inhibitor are administered peritumorally.

In certain embodiments, the first antibody, the human PD-1 inhibitor, or the human CTLA-4 inhibitor is administered by intravenous infusion over about 30 minutes. In certain embodiments, the first antibody, the human PD-1 inhibitor, or the human CTLA-4 inhibitor is administered by intravenous infusion over about 45 minutes. In certain embodiments, the first antibody, the human PD-1 inhibitor, or the human CTLA-4 inhibitor is administered by intravenous infusion over about 60 minutes. In certain embodiments, the first antibody, the human PD-1 inhibitor, or the human CTLA-4 inhibitor is administered by intravenous infusion over about 90 minutes.

In certain embodiments, the first antibody and the human PD-1 inhibitor are each administered by intravenous infusion over about 30 minutes. In certain embodiments, the first antibody and the human PD-1 inhibitor are each administered by intravenous infusion over about 45 minutes. In certain embodiments, the first antibody and the human PD-1 inhibitor are each administered by intravenous infusion over about 60 minutes. In certain embodiments, the first antibody and the human PD-1 inhibitor are each administered by intravenous infusion over about 90 minutes. In certain embodiments, the first antibody and the human CTLA-4 inhibitor are each administered by intravenous infusion over about 30 minutes. In certain embodiments, the first antibody and the human CTLA-4 inhibitor are each administered by intravenous infusion over about 45 minutes. In certain embodiments, the first antibody and the human CTLA-4 inhibitor are each administered by intravenous infusion over about 60 minutes. In certain embodiments, the first antibody and the human CTLA-4 inhibitor are each administered by intravenous infusion over about 90 minutes. In certain embodiments, the first antibody, the human PD-1 inhibitor, and the human CTLA-4 inhibitor are each administered by intravenous infusion over about 30 minutes. In certain embodiments, the first antibody, the human PD-1 inhibitor, and the human CTLA-4 inhibitor are each administered by intravenous infusion over about 45 minutes. In certain embodiments, the first antibody, the human PD-1 inhibitor, and the human CTLA-4 inhibitor are each administered by intravenous infusion over about 60 minutes. In certain embodiments, the first antibody, the human PD-1 inhibitor, and the human CTLA-4 inhibitor are each administered by intravenous infusion over about 90 minutes.

In certain embodiments, the human PD-1 inhibitor is administered to the subject subsequent to administration of the first antibody. In certain embodiments, the human CTLA-4 inhibitor is administered to the subject subsequent to administration of the first antibody. In certain embodiments, the human CTLA-4 inhibitor is administered to the subject subsequent to administration of the first antibody and the human PD-1 inhibitor.

In certain embodiments, the dose of the first antibody and the dose of the human PD-1 inhibitor are administered on the same day. In certain embodiments, the dose of the first antibody and the dose of the human PD-1 inhibitor are administered simultaneously. In certain embodiments, the dose of the first antibody is administered prior to (e.g., 30 minutes before, 1 hour before, 2 hours before, 4 hours before, 8 hours before, 12 hours before, 16 hours before, 20 hours before, 1 day before, 1.5 days before, 2 days before, 3 days before, etc.) the dose of the human PD-1 inhibitor. In certain embodiments, the dose of the first antibody is administered after (e.g., 30 minutes after, 1 hour after, 2 hours after, 4 hours after, 8 hours after, 12 hours after, 16 hours after, 20 hours after, 1 day after, 1.5 days after, 2 days after, 3 days after, etc.) the dose of the human PD-1 inhibitor.

In certain embodiments, the dose of the first antibody and the dose of the human CTLA-4 inhibitor are administered on the same day. In certain embodiments, the dose of the first antibody and the dose of the human CTLA-4 inhibitor are administered simultaneously. In certain embodiments, the dose of the first antibody is administered prior to (e.g., 30 minutes before, 1 hour before, 2 hours before, 4 hours before, 8 hours before, 12 hours before, 16 hours before, 20 hours before, 1 day before, 1.5 days before, 2 days before, 3 days before, etc.) the dose of the human CTLA-4 inhibitor. In certain embodiments, the dose of the first antibody is administered after (e.g., 30 minutes after, 1 hour after, 2 hours after, 4 hours after, 8 hours after, 12 hours after, 16 hours after, 20 hours after, 1 day after, 1.5 days after, 2 days after, 3 days after, etc.) the dose of the human CTLA-4 inhibitor.

In certain embodiments, the dose of the first antibody, the dose of the human PD-1 inhibitor, and the dose of the human CTLA-4 inhibitor are administered on the same day. In certain embodiments, the dose of the first antibody, the dose of the human PD-1 inhibitor, and the dose of the human CTLA-4 inhibitor are administered simultaneously. In certain embodiments, the dose of the first antibody is administered prior to (e.g., 30 minutes before, 1 hour before, 2 hours before, 4 hours before, 8 hours before, 12 hours before, 16 hours before, 20 hours before, 1 day before, 1.5 days before, 2 days before, 3 days before, etc.) the dose of the human PD-1 inhibitor, which is administered prior to (e.g., 30 minutes before, 1 hour before, 2 hours before, 4 hours before, 8 hours before, 12 hours before, 16 hours before, 20 hours before, 1 day before, 1.5 days before, 2 days before, 3 days before, etc.) the dose of the human CTLA-4 inhibitor.

In certain embodiments, the dose of the first antibody is a therapeutically effective amount. In certain embodiments, dose of the first antibody and the dose of the human PD-1 inhibitor is a therapeutically effective amount. In certain embodiments, dose of the first antibody and the dose of the human CTLA-4 inhibitor is a therapeutically effective amount. In certain embodiments, the dose of the first antibody, the dose of the human PD-1 inhibitor, and the dose of the human CTLA-4 inhibitor is a therapeutically effective amount.

Any disease or disorder in a subject that would benefit from inhibition of ILT2, CTLA-4, and/or PD-1 function can be treated using the methods, antibodies, or inhibitors described herein. The methods, antibodies, or inhibitors described herein are particularly useful for inhibiting immune system tolerance to tumors, and accordingly can be used as an immunotherapy for subjects with cancer. For example, in certain embodiments, the present disclosure provides a method of treating cancer in a subject, the method comprising administering to the subject an effective amount of an antibody and/or inhibitor as described herein.

In certain embodiments, the cancer treated in accordance with the methods described herein is a metastatic or locally advanced cancer (e.g., solid tumor). In certain embodiments, the cancer is treated in accordance with a method described herein as a first cancer therapy after diagnosis of the metastatic or locally advanced tumor (e.g., within 1, 2, 3, 4, 5, or 6 days; 1, 2, 3, 4, 6, 8, or 12 weeks; or, 1, 2, 3, 4, 6, 8, or 12 months after diagnosis). In certain embodiments, the cancer is treated in accordance with a method described herein as the first cancer therapy after diagnosis of tumor progression (e.g., within 1, 2, 3, 4, 5, or 6 days; 1, 2, 3, 4, 6, 8, or 12 weeks; or, 1, 2, 3, 4, 6, 8, or 12 months after diagnosis of tumor progression) that has occurred despite previous treatment of the tumor with a different cancer therapy, optionally wherein the method described herein is provided as the second cancer therapy administered. In certain embodiments, the cancer is treated in accordance with a method described herein as the first cancer therapy after diagnosis of toxicity of a different cancer therapy (e.g., within 1, 2, 3, 4, 5, or 6 days; 1, 2, 3, 4, 6, 8, or 12 weeks; or, 1, 2, 3, 4, 6, 8, or 12 months after diagnosis of toxicity of the different cancer therapy), optionally wherein the method described herein is provided as the second cancer therapy administered. In certain embodiments, the cancer treated in accordance with the methods described herein is a metastatic or locally advanced cancer (e.g., solid tumor) for which no standard therapy is available. In other embodiments, the cancer treated in accordance with the methods described herein is a metastatic or locally advanced cancer (e.g., solid tumor) for which a standard therapy has failed (i.e., the cancer has progressed after the standard therapy). In certain embodiments, a therapy fails if the cancer is refractory to the therapy. In certain embodiments, a therapy fails if the cancer relapses after responding, fully or partially, to the therapy. In certain embodiments, metastatic or locally advanced cancer (e.g., solid tumor) has been confirmed histologically or cytologically.

In certain embodiments, the cancer is a solid tumor. In certain embodiments, the cancer (e.g., solid tumor) expresses ILT2. In certain embodiments, the metastatic or locally advanced cancer (e.g., solid tumor) expresses ILT2. In certain embodiments, the cancer (e.g., solid tumor) expresses PD-1. In certain embodiments, the metastatic or locally advanced cancer (e.g., solid tumor) expresses PD-1. In certain embodiments, the cancer (e.g., solid tumor) expresses PD-L1. In certain embodiments, the metastatic or locally advanced cancer (e.g., solid tumor) expresses PD-L1. In certain embodiments, the cancer (e.g., solid tumor) expresses CTLA-4. In certain embodiments, the metastatic or locally advanced cancer (e.g., solid tumor) expresses CTLA-4.

Cancers that can be treated according to the methods described herein include, without limitation, solid cancer (e.g., relapsed or refractory solid cancer, and advanced or metastatic solid cancer), carcinoma, sarcoma, melanoma (e.g., stage III or stage IV melanoma), small cell lung cancer, non-small cell lung cancer, urothelial cancer, ovarian cancer, prostate cancer (e.g., metastatic hormone-refractory prostate cancer and progressive metastatic prostate cancer), pancreatic cancer, breast cancer (e.g., HER2⁺ breast cancer (e.g., relapsed/refractory HER2+ breast cancer)), head and neck cancer (e.g., relapsed/refractory head and neck squamous cell carcinoma (HNSCC)), glioma, malignant glioma, glioblastoma multiforme, brain metastasis, merkel cancer, gastric cancer, gastroesophageal cancer, renal cell carcinoma, uveal melanoma, colon cancer, cervical cancer, lymphoma (e.g., relapsed or refractory lymphoma), non-Hodgkin's lymphoma, Hodgkin's lymphoma, leukemia, and multiple myeloma.

In certain embodiments, the cancer treated in accordance with the methods described herein is B cell lymphoma (e.g., B cell chronic lymphocytic leukemia, B cell non-Hodgkin lymphoma, cutaneous B cell lymphoma, diffuse large B cell lymphoma), basal cell carcinoma, bladder cancer, blastoma, brain metastasis, breast cancer, Burkitt lymphoma, carcinoma (e.g., adenocarcinoma (e.g., of the gastroesophageal junction)), cervical cancer, colon cancer, colorectal cancer (colon cancer and rectal cancer), endometrial carcinoma, esophageal cancer, Ewing sarcoma, follicular lymphoma, gastric cancer, gastroesophageal junction carcinoma, gastrointestinal cancer, glioblastoma (e.g., glioblastoma multiforme, e.g., newly diagnosed or recurrent), glioma, head and neck cancer (e.g., head and neck squamous cell carcinoma), hepatic metastasis, Hodgkin's and non-Hodgkin's lymphoma, kidney cancer (e.g., renal cell carcinoma and Wilms' tumors), laryngeal cancer, leukemia (e.g., chronic myelocytic leukemia, hairy cell leukemia), liver cancer (e.g., hepatic carcinoma and hepatoma), lung cancer (e.g., non-small cell lung cancer and small-cell lung cancer), lymphoblastic lymphoma, lymphoma, mantle cell lymphoma, metastatic brain tumor, metastatic cancer, myeloma (e.g., multiple myeloma), neuroblastoma, ocular melanoma, oropharyngeal cancer, osteosarcoma, ovarian cancer, pancreatic cancer (e.g., pancreatic ductal adenocarcinoma), prostate cancer (e.g., hormone refractory (e.g., castration resistant), metastatic, metastatic hormone refractory (e.g., castration resistant, androgen independent)), renal cell carcinoma (e.g., metastatic), salivary gland carcinoma, sarcoma (e.g., rhabdomyosarcoma), skin cancer (e.g., melanoma (e.g., metastatic melanoma)), soft tissue sarcoma, solid tumor, squamous cell carcinoma, synovia sarcoma, testicular cancer, thyroid cancer, transitional cell cancer (urothelial cell cancer), uveal melanoma (e.g., metastatic), verrucous carcinoma, vulval cancer, and Waldenstrom macroglobulinemia.

In certain embodiments, the cancer treated in accordance with the methods described herein is human sarcoma or carcinoma, e.g., fibrosarcoma, myxosarcoma, liposarcoma, chondrosarcoma, sarcoma, osteogenic chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma (e.g., metastatic), hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, melanoma, neuroblastoma, or retinoblastoma.

In certain embodiments, the cancer treated in accordance with the methods described herein is an acute lymphocytic leukemia or acute myelocytic leukemia (e.g., myeloblastic, promyelocytic, myelomonocytic, monocytic and erythroleukemia); chronic leukemia (chronic myelocytic (granulocytic) leukemia or chronic lymphocytic leukemia); Hodgkin's disease; non-Hodgkin's disease; acute myeloid leukemia; B-cell lymphoma; T-cell lymphoma; anaplastic large cell lymphoma; intraocular lymphoma; follicular lymphoma; small intestine lymphoma; or splenic marginal zone lymphoma.

In certain embodiments, the cancer treated in accordance with the methods described herein is multiple myeloma, Waldenstrom's macroglobulinemia, heavy chain disease, gastrointestinal stromal tumors, head and/or neck cancer (e.g., squamous cell carcinoma of the hypopharynx, squamous cell carcinoma of the larynx, cell carcinoma of the oropharynx, or verrucous carcinoma of the larynx), endometrial stromal sarcoma, mast cell sarcoma, adult soft tissue sarcoma, uterine sarcoma, merkel cell carcinoma, urothelial carcinoma, melanoma with brain metastases, uveal melanoma, uveal melanoma with liver metastases, non-small cell lung cancer, rectal cancer, or myelodysplastic syndrome. In some embodiments, the cancer treated in accordance with the methods is metastatic.

In certain embodiments, the cancer treated in accordance with the methods described herein is prostate cancer, breast cancer, lung cancer, colorectal cancer, melanoma, bronchial cancer, bladder cancer, brain or central nervous system cancer, peripheral nervous system cancer, uterine or endometrial cancer, cancer of the oral cavity or pharynx, non-Hodgkin's lymphoma, thyroid cancer, kidney cancer, biliary tract cancer, small bowel or appendix cancer, salivary gland cancer, thyroid gland cancer, adrenal gland cancer, squamous cell cancer, mesothelioma, osteocarcinoma, thyoma/thymic carcinoma, glioblastoma, myelodysplastic syndrome, soft tissue sarcoma, DIPG, adenocarcinoma, osteosarcoma, chondrosarcoma, leukemia, or pancreatic cancer. In some embodiments, the cancer treated in accordance with the methods described herein includes a carcinoma (e.g., an adenocarcinoma), lymphoma, blastoma, melanoma, sarcoma, or leukemia.

In certain embodiments, the cancer treated in accordance with the methods described herein is squamous cell cancer, small-cell lung cancer, non-small cell lung cancer, gastrointestinal cancer, Hodgkin's lymphoma, non-Hodgkin's lymphoma, pancreatic cancer, glioblastoma, glioma, cervical cancer, ovarian cancer, liver cancer (e.g., hepatic carcinoma and hepatoma), bladder cancer, breast cancer, inflammatory breast cancer, Merkel cell carcinoma, colon cancer, colorectal cancer, stomach cancer, urinary bladder cancer, endometrial carcinoma, myeloma (e.g., multiple myeloma), salivary gland, carcinoma, kidney cancer (e.g., renal cell carcinoma and Wilms' tumors), basal cell carcinoma, melanoma, prostate cancer, vulval cancer, thyroid cancer, testicular cancer, esophageal cancer, serous adenocarcinoma or various types of head and neck cancer. In certain embodiments, the cancer treated in accordance with the methods described herein includes desmoplastic melanoma, inflammatory breast cancer, thymoma, rectal cancer, anal cancer, or surgically treatable or non-surgically treatable brain stem glioma.

In certain embodiments, the cancer treated in accordance with the methods described herein is metastatic melanoma (e.g., resistant metastatic melanoma), metastatic ovarian cancer, or metastatic renal cell carcinoma. In certain embodiments, the cancer treated in accordance with the methods described herein is melanoma that is resistant to ipilimumab. In some embodiments, the cancer treated in accordance with the methods described herein is melanoma that is resistant to nivolumab or pembrolizumab. In some embodiments, the cancer treated in accordance with the methods described herein is melanoma that is resistant to ipilimumab and nivolumab or pembrolizumab.

In certain embodiments, the cancer treated in accordance with the methods described herein is breast cancer (e.g., herceptin resistant breast cancer and trastuzumab-DM1 (T-DM1) resistant breast cancer), prostate cancer, glioblastoma multiforme, colorectal cancer, sarcoma, bladder cancer, cervical cancer, HPV-associated cancers, cancers of the vagina, cancers of the vulva, cancers of the penis, cancer of the anus, cancer of the rectum, cancer of the oropharynx, multiple myeloma, renal cell carcinoma, ovarian cancer, hepatocellular cancer, endometrial cancer, pancreatic cancer, lymphoma, and leukemia (e.g., elderly leukemia, acute myeloid leukemia (AML), and elderly AML).

In certain embodiments, the cancer treated in accordance with the methods described herein is metastatic malignant melanoma (e.g., cutaneous or intraocular malignant melanoma), renal cancer (e.g., clear cell carcinoma), prostate cancer (e.g., hormone refractory prostate adenocarcinoma), breast cancer, colon cancer, lung cancer (e.g., non-small cell lung cancer), bone cancer, pancreatic cancer, skin cancer, cancer of the head or neck, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, glioma, pituitary adenoma, Kaposi's sarcoma, epidermoid cancer, squamous cell cancer, T-cell lymphoma, environmentally induced cancers including those induced by asbestos, esophageal cancer, liver cancer, refractory or recurrent malignancies, metastatic cancers, and combinations of said cancers.

In certain embodiments, the subject has previously received an immunotherapy. In certain embodiments, the subject has not previously received any immunotherapy. In certain embodiments, the cancer is an advanced or metastatic cancer.

In certain embodiments, the subject is at least 18 years of age. In certain embodiments, the subject has a histologically confirmed diagnosis of a solid tumor that is currently metastatic or locally advanced. In certain embodiments, no standard therapy is available or standard therapy has failed. In certain embodiments, before administration of the first antibody and/or the human PD-1 inhibitor and/or the human CTLA-4 inhibitor, the subject has measurable disease on baseline imaging as assessed according to Response Evaluation Criteria in Solid Tumors Version 1.1 (RECIST 1.1). In certain embodiments, the subject has a predicted life expectancy of ≥3 months. In certain embodiments, before administration of the first antibody and/or the human PD-1 inhibitor and/or the human CTLA-4 inhibitor, the subject has an Eastern Cooperative Oncology Group performance status of 0-1. In certain embodiments, before administration of the first antibody and/or the human PD-1 inhibitor and/or the human CTLA-4 inhibitor, the subject has adequate organ and bone marrow reserve function as defined by one or more of: absolute neutrophil count ≥1.5×10⁹/L; platelet count ≥100×10⁹/L; hemoglobin ≥8.0 g/dL without transfusion within 2 weeks prior to hemoglobin measurement; serum albumin ≥3.0 g/dL; total bilirubin ≤1.5× upper limit of normal (ULN); aspartate aminotransferase ≤2.5×ULN; alanine aminotransferase ≤2.5×ULN; alkaline phosphatase ≤2.5×ULN, or ≤5.0×ULN for subjects with liver metastases; creatinine clearance ≥40 mL/min, as assessed by Cockcroft-Gault method (see Cockcroft D W, Gault MH. Nephron. 1976; 16(1):31-41); and/or international normalized ratio or prothrombin time 23 1.5×ULN and activated partial thromboplastin time ≤1.5×ULN, unless patient is receiving anticoagulant therapy. In certain embodiments, the subject is not pregnant and/or is not breastfeeding.

In certain embodiments, the subject the subject has not had a cytotoxic therapy or other monoclonal antibody therapy within 3 weeks prior to administration of the first antibody and/or the human PD-1 inhibitor and/or the human CTLA-4 inhibitor. In certain embodiments, the subject has not had radiation therapy to the central nervous system (CNS) within 2 weeks prior to, or non-CNS radiation therapy within one week prior to administration of the first antibody and/or the human PD-1 inhibitor and/or the human CTLA-4 inhibitor. In certain embodiments, the subject has not had small molecule targeted therapy or tyrosine kinase inhibitor therapy within 2 weeks prior to administration of the first antibody and/or the human PD-1 inhibitor and/or the human CTLA-4 inhibitor, or 5 half-lives of the therapy.

In certain embodiments, the subject does not have any persistent toxicities (Common Terminology Criteria for Adverse Events [CTCAE]≥1) from prior cancer therapies. In certain embodiments, the subject does not have sensory neuropathy or alopecia of Grade >2. In certain embodiments, the subject does not have a history of or active interstitial lung disease. In certain embodiments, the subject does not have a history of anaphylaxis or uncontrolled asthma. In certain embodiments, the subject does not have a condition requiring systemic treatment with either corticosteroids (>10 mg daily prednisone equivalent) within 2 weeks prior to or other immunosuppressive medication within 30 days prior to administration of the first antibody and/or the human PD-1 inhibitor and/or the human CTLA-4 inhibitor. In certain embodiments, the subject does not have active CNS metastases, unless the CNS metastases has been treated and the subject is radiologically and clinically stable. In certain embodiments, the subject does not have active or history of autoimmune disease that requires systemic treatment within 2 years prior to administration of the first antibody and/or the human PD-1 inhibitor and/or the human CTLA-4 inhibitor, unless treatment consists of hormone replacement therapy or topical treatment. In certain embodiments, the subject has not had an allogeneic tissue or solid organ transplant, except for a corneal transplant.

In certain embodiments, the subject has not had an active infection requiring treatment within 2 weeks prior to administration of the first antibody and/or the human PD-1 inhibitor and/or the human CTLA-4 inhibitor. In certain embodiments, the subject is not HIV positive, unless the subject has CD4>200 cells/mm³ or an undetectable viral load. In certain embodiments, the subject does not have active hepatitis B (HBV) or hepatitis C (HPC), unless hepatitis B surface antigen and HBV DNA, or HCV RNA, respectively, are negative. In certain embodiments, the subject does not have clinically significant cardiovascular disease, as defined by one or more of: cerebral vascular accident or stroke or myocardial infarction within 6 months prior to administration of the first antibody and/or the human PD-1 inhibitor and/or the human CTLA-4 inhibitor; unstable angina; congestive heart failure (New York Heart Association class ≥II); and/or uncontrolled cardiac arrhythmia requiring medication. In certain embodiments, the subject does not have QTc interval >480 msec, unless prolonged QTc is due to right bundle branch block. In certain embodiments, the subject does not have uncontrolled hypertension (>140/90 mmHg), or controlled hypertension on more than 3 antihypertensive agents.

In certain embodiments, the first antibody is administered as a monotherapy. In certain embodiments, the first antibody is administered on the same day as the human PD-1 inhibitor. In certain embodiments, the first antibody is administered on the same day as the human CTLA-4 inhibitor. In certain embodiments, the first antibody is administered on the same day as the human PD-1 inhibitor and the human CTLA-4 inhibitor. In certain embodiments, each administration of the first antibody is on the same day as the human PD-1 inhibitor. In certain embodiments, each administration of the human CTLA-4 inhibitor is on the same day as the first antibody. In certain embodiments, the human PD-1 inhibitor is administered within 30 minutes or 1 hour after the completion of administration of the first antibody. In certain embodiments, the human CTLA-4 inhibitor is administered within 30 minutes or 1 hour after the completion of administration of the first antibody. In certain embodiments, the human CTLA-4 inhibitor is administered within 30 minutes or 1 hour after the completion of administration of the human PD-1 inhibitor.

In certain embodiments, the first antibody is administered at 1 mg to 1000 mg and the human PD-1 inhibitor is administered at 450 mg. In certain embodiments, the first antibody is administered at 1 mg and the human PD-1 inhibitor is administered at 450 mg. In certain embodiments, the first antibody is administered at 3 mg and the human PD-1 inhibitor is administered at 450 mg. In certain embodiments, the first antibody is administered at 10 mg and the human PD-1 inhibitor is administered at 450 mg. In certain embodiments, the first antibody is administered at 30 mg and the human PD-1 inhibitor is administered at 450 mg. In certain embodiments, the first antibody is administered at 100 mg and the human PD-1 inhibitor is administered at 450 mg. In certain embodiments, the first antibody is administered at 300 mg and the human PD-1 inhibitor is administered at 450 mg. In certain embodiments, the first antibody is administered at 1000 mg and the human PD-1 inhibitor is administered at 450 mg.

In certain embodiments, the first antibody is administered at 1 mg to 1000 mg and the human CTLA-4 inhibitor is administered at 150 mg. In certain embodiments, the first antibody is administered at 1 mg and the human CTLA-4 inhibitor is administered at 150 mg. In certain embodiments, the first antibody is administered at 3 mg and the human CTLA-4 inhibitor is administered at 150 mg. In certain embodiments, the first antibody is administered at 10 mg and the human CTLA-4 inhibitor is administered at 150 mg. In certain embodiments, the first antibody is administered at 30 mg and the human CTLA-4 inhibitor is administered at 150 mg. In certain embodiments, the first antibody is administered at 100 mg and the human CTLA-4 inhibitor is administered at 150 mg. In certain embodiments, the first antibody is administered at 300 mg and the human CTLA-4 inhibitor is administered at 150 mg. In certain embodiments, the first antibody is administered at 1000 mg and the human CTLA-4 inhibitor is administered at 150 mg.

In certain embodiments, the first antibody is administered at 1 mg to 1000 mg, the human PD-1 inhibitor is administered at 450 mg, and the human CTLA-4 inhibitor is administered at 150 mg. In certain embodiments, the first antibody is administered at 1 mg, the human PD-1 inhibitor is administered at 450 mg, and the human CTLA-4 inhibitor is administered at 150 mg. In certain embodiments, the first antibody is administered at 3 mg, the human PD-1 inhibitor is administered at 450 mg, and the human CTLA-4 inhibitor is administered at 150 mg. In certain embodiments, the first antibody is administered at 10 mg, the human PD-1 inhibitor is administered at 450 mg, and the human CTLA-4 inhibitor is administered at 150 mg. In certain embodiments, the first antibody is administered at 30 mg, the human PD-1 inhibitor is administered at 450 mg, and the human CTLA-4 inhibitor is administered at 150 mg. In certain embodiments, the first antibody is administered at 100 mg, the human PD-1 inhibitor is administered at 450 mg, and the human CTLA-4 inhibitor is administered at 150 mg. In certain embodiments, the first antibody is administered at 300 mg, the human PD-1 inhibitor is administered at 450 mg, and the human CTLA-4 inhibitor is administered at 150 mg. In certain embodiments, the first antibody is administered at 1000 mg, the human PD-1 inhibitor is administered at 450 mg, and the human CTLA-4 inhibitor is administered at 150 mg.

The first antibody, human PD-1 inhibitor, and human CTLA-4 inhibitor can be administered simultaneously or sequentially by the same or different routes of administration (e.g., as described herein). In certain embodiments, the first antibody, human PD-1 inhibitor, and human CTLA-4 inhibitor are administered simultaneously via intravenous injection (e.g., intravenous infusion). In certain embodiments, the first antibody, human PD-1 inhibitor, and human CTLA-4 inhibitor are administered sequentially via intravenous injection (e.g., intravenous infusion). In certain embodiments, the first antibody is administered intravenously at 1 mg to 1000 mg and the human PD-1 inhibitor is administered intravenously at 450 mg, every three weeks. In certain embodiments, the first antibody is administered intravenously at 1 mg to 1000 mg and the human CTLA-4 inhibitor is administered intravenously at 150 mg, every three weeks. In certain embodiments, the first antibody is administered intravenously at 1 mg to 1000 mg every 3 weeks, the human PD-1 inhibitor is administered intravenously at 450 mg every three weeks, and the human CTLA-4 inhibitor is administered at 150 mg every 6 weeks.

In certain embodiments, the method comprises administering to the subject a first antibody, e.g., an antibody comprising a heavy chain and a light chain comprising the amino acid sequences of SEQ ID NOs: 9 and 10, respectively; a human PD-1 inhibitor, e.g., balstilimab; and a human CTLA-4 inhibitor, e.g., botensilimab. In certain embodiments, balstilimab is administered at a dose of 450 mg once every 3 weeks by intravenous infusion. In certain embodiments, botensilimab is administered at a dose of 150 mg once every 6 weeks by intravenous infusion.

In certain embodiments, balstilimab is administered at a dose of 450 mg once every 3 weeks by intravenous infusion, botensilimab is administered at a dose of 150 mg once every 6 weeks by intravenous infusion, and the first antibody is administered at a dose of about 1 mg to about 2000 mg, about 1 mg to about 1000 mg, about 3 mg to about 300 mg, about 10 mg to about 100 mg, about 1 mg to about 600 mg, about 1 mg to about 300 mg, about 1 mg to about 100 mg, about 1 mg to about 30 mg, about 1 mg to about 10 mg, about 1 mg to about 3 mg, about 10 mg to about 2000 mg, about 10 mg to about 1000 mg, about 10 mg to about 600 mg, about 10 mg to about 300 mg, about 10 mg to about 30 mg, about 30 mg to about 1000 mg, about 30 mg to about 600 mg, about 30 mg to about 300 mg, about 30 mg to about 100 mg, about 100 mg to about 1000 mg, about 100 mg to about 600 mg, about 100 mg to about 300 mg, about 300 mg to about 2000 mg, about 300 mg to about 1000 mg, or about 300 mg to about 600 mg. In certain embodiments, balstilimab is administered at a dose of 450 mg once every 3 weeks by intravenous infusion, botensilimab is administered at a dose of 150 mg once every 6 weeks by intravenous infusion, and the first antibody is administered at a dose of about 1 mg, about 3 mg, about 10 mg, about 30 mg, about 100 mg, about 300 mg, about 400, about 500, about 600, about 700. about 800, about 900, about 1000 mg, about 1250, about 1500, about 1750, or about 2000.

In certain embodiments, balstilimab is administered at a dose of 450 mg once every 3 weeks by intravenous infusion, botensilimab is administered at a dose of 150 mg once every 6 weeks by intravenous infusion, and the first antibody is administered intravenously. In certain embodiments, balstilimab is administered at a dose of 450 mg once every 3 weeks by intravenous infusion, botensilimab is administered at a dose of 150 mg once every 6 weeks by intravenous infusion, and the first antibody is administered by intravenous infusion over about 30 minutes.

In certain embodiments, balstilimab is administered at a dose of 450 mg once every 3 weeks by intravenous infusion, botensilimab is administered at a dose of 150 mg once every 6 weeks by intravenous infusion, and the first antibody is administered once weekly, once every 2 weeks, once every 3 weeks, once every 4 weeks, once every 5 weeks, or once every 6 weeks. In certain embodiments, balstilimab is administered at a dose of 450 mg once every 3 weeks by intravenous infusion, botensilimab is administered at a dose of 150 mg once every 6 weeks by intravenous infusion, and the first antibody is administered intravenously at a dose of 1 mg once every 3 weeks. In certain embodiments, balstilimab is administered at a dose of 450 mg once every 3 weeks by intravenous infusion, botensilimab is administered at a dose of 150 mg once every 6 weeks by intravenous infusion, and the first antibody is administered intravenously at a dose of 3 mg once every 3 weeks. In certain embodiments, balstilimab is administered at a dose of 450 mg once every 3 weeks by intravenous infusion, botensilimab is administered at a dose of 150 mg once every 6 weeks by intravenous infusion, and the first antibody is administered intravenously at a dose of 10 mg once every 3 weeks. In certain embodiments, balstilimab is administered at a dose of 450 mg once every 3 weeks by intravenous infusion, botensilimab is administered at a dose of 150 mg once every 6 weeks by intravenous infusion, and the first antibody is administered intravenously at a dose of 30 mg once every 3 weeks. In certain embodiments, balstilimab is administered at a dose of 450 mg once every 3 weeks by intravenous infusion, botensilimab is administered at a dose of 150 mg once every 6 weeks by intravenous infusion, and the first antibody is administered intravenously at a dose of 100 mg once every 3 weeks. In certain embodiments, balstilimab is administered at a dose of 450 mg once every 3 weeks by intravenous infusion, botensilimab is administered at a dose of 150 mg once every 6 weeks by intravenous infusion, and the first antibody is administered intravenously at a dose of 300 mg once every 3 weeks. In certain embodiments, balstilimab is administered at a dose of 450 mg once every 3 weeks by intravenous infusion, botensilimab is administered at a dose of 150 mg once every 6 weeks by intravenous infusion, and the first antibody is administered intravenously at a dose of 1000 mg once every 3 weeks.

In an aspect, provided herein is an antibody that specifically binds to human ILT2 for use in the treatment of cancer, wherein the treatment is performed according to a method described herein. In an aspect, provided herein is an antibody that specifically binds to human ILT2 and a human PD-1 inhibitor for use in the treatment of cancer, wherein the treatment is performed according to a method described herein. In an aspect, provided herein is an antibody that specifically binds to human ILT2 and a human CTLA-4 inhibitor for use in the treatment of cancer, wherein the treatment is performed according to a method described herein. In an aspect, provided herein is an antibody that specifically binds to human ILT2, a human PD-1 inhibitor, and a human CTLA-4 inhibitor for use in the treatment of cancer, wherein the treatment is performed according to a method described herein.

In an aspect, provided herein is an antibody that specifically binds to human ILT2 for use in the manufacture of a medicament for the treatment of cancer, wherein the treatment is performed according to a method described herein. In an aspect, provided herein is an antibody that specifically binds to human ILT2 and a human PD-1 inhibitor for use in the manufacture of a medicament for the treatment of cancer, wherein the treatment is performed according to a method described herein. In an aspect, provided herein is an antibody that specifically binds to human ILT2 and a human CTLA-4 inhibitor for use in the manufacture of a medicament for the treatment of cancer, wherein the treatment is performed according to a method described herein. In an aspect, provided herein is an antibody that specifically binds to human ILT2, a human PD-1 inhibitor, and a human CTLA-4 inhibitor for use in the manufacture of a medicament for the treatment of cancer, wherein the treatment is performed according to a method described herein.

In an aspect, provided herein is an antibody that specifically binds to human ILT2 for the treatment of cancer, wherein the treatment is performed according to a method described herein. In an aspect, provided herein is an antibody that specifically binds to human ILT2 and a human PD-1 inhibitor for the treatment of cancer, wherein the treatment is performed according to a method described herein. In an aspect, provided herein is an antibody that specifically binds to human ILT2 and a human CTLA-4 inhibitor for the treatment of cancer, wherein the treatment is performed according to a method described herein. In an aspect, provided herein is an antibody that specifically binds to human ILT2, a human PD-1 inhibitor, and a human CTLA-4 inhibitor for the treatment of cancer, wherein the treatment is performed according to a method described herein.

### EXAMPLES

The examples in this section are offered by way of illustration, and not by way of limitation.

### Example 1: A Phase 1 Study Investigating AGEN1571 as a Single Agent and in Combination with a PD-1 Inhibitor and/or Botensilimab (AGEN1181) in Patients with Advanced Solid Tumors

AGEN1571 is a fully human monoclonal IgG₄ antibody that antagonizes ILT2. This Example describes a multicenter, Phase 1 trial to evaluate the safety, tolerability, PK, and pharmacodynamics of AGEN1571 as a single agent and when used in combination with balstilimab and/or botensilimab in patients with advanced or metastatic solid tumors to determine the recommended Phase 2 dose (RP2D).

### Overall Study Design

This is an open-label, Phase 1, 2-part trial to determine RP2Ds and to evaluate the safety, tolerability, PK and pharmacodynamic profiles of AGEN1571 as a monotherapy and in combination with balstilimab and/or botensilimab in patients diagnosed with advanced solid tumors.

The study will enroll up to 98 evaluable patients. The actual number of patients enrolled will be driven by safety, tolerability, and duration of treatment.

Part 1 is a dose escalation phase to determine the RP2D of AGEN1571 monotherapy or AGEN1571 in combination with balstilimab or botensilimab (2-agent combinations) or in combination with both balstilimab and botensilimab (3-agent combination).

Part 2 of the trial is a dose expansion phase for specific disease indications.

### Objectives and Endpoints

TABLE 4
Objectives and Endpoints

| | |
|---|---|
| Objectives | Endpoints |

Primary

| | |
|---|---|
| To determine the recommended | Occurrence of dose-limiting toxicities |
| Phase 2 dose (RP2D) of AGEN1571 | (DLTs) within 42 days after the first |
| as a single agent and in combination | administration of AGEN1571 as a single |
| with balstilimab and/or | agent and in combination with balstilimab |
| botensilimab. | and/or botensilimab. |
| To evaluate the safety and | Frequency and severity of adverse events |
| tolerability of AGEN1571 as a | (AEs), and duration of treatment-emergent |
| single agent and in combination with | adverse events (TEAEs) according to |
| balstilimab and/or botensilimab. | National Cancer Institute Common |
| | Terminology Criteria for Adverse Events |
| | (NCI CTCAE) Version 5.0, vital signs |
| | (blood pressure, heart rate, and |
| | temperature), physical examinations, 12- |
| | lead electrocardiogram (ECG), Eastern |
| | Cooperative Oncology Group (ECOG) |
| | performance status, and clinical laboratory |
| | assessments for all dose groups. |

Secondary

| | |
|---|---|
| To characterize the | Serum AGEN1571, balstilimab, and |
| pharmacokinetics (PK) when | botensilimab concentrations |
| AGEN1571 is administered as a | Serum AGEN1571, balstilimab, and |
| single agent and in combination with | botensilimab anti-drug antibody (ADA) |
| balstilimab and/or botensilimab. | determinations |
| To evaluate immunogenicity of | Objective response rate (ORR) including |
| AGEN1571 as a single agent and in | complete response (CR) and partial |
| combination with balstilimab and/or | response (PR) rates, duration of response |
| botensilimab and assess potential | (DOR), disease control rate (DCR) and |
| impact on PK and biological | progression-free survival (PFS) per |
| activity. | Response Evaluation Criteria in Solid |
| To assess efficacy of AGEN1571 as | Tumors Version 1.1 (RECIST 1.1) based |
| a single agent or in combination | on Investigator assessment |
| with balstilimab and/or botensilimab | |

Exploratory

| | |
|---|---|
| To explore biomarkers that may | Associated measures between systemic |
| predict pharmacologic activity or | AGEN1571 PK measures and select |
| response to AGEN1571 as a single | biomarkers of interest |
| agent and when combined with | Biomarker endpoints may include, but are |
| balstilimab and/or botensilimab | not limited to, analysis of the following |
| To evaluate potential biomarkers | potential markers of disease or the drug |
| that may predict pharmacologic | mechanism of action measured on tumor |
| activity or response to AGEN1571 | and/or peripheral blood samples prior to or |
| as monotherapy and when combined | following treatment: |
| with balstilimab and/or botensilimab | Genetic markers (DNA and RNA- |
| To further assess preliminary | based) |
| efficacy of AGEN1571 as a single | Expression/presence of proteins |
| agent or in combination with | (soluble or cell membrane |
| balstilimab and/or botensilimab | expressed) |
| | Immune cell presence and activity |
| | Overall survival is defined as the first dose |
| | of study drug to the date of death due to |
| | any cause. |

### Study Treatments

### Part 1—Dose Escalation Phase

Part 1 of the study will be composed of 4 study treatment arms with patients assigned using an accelerated titration design for AGEN1571 monotherapy for Cohorts 1 to 3 followed by a traditional 3+3 design for Cohorts 4 to 7 for Study Treatment Arm 1 (AGEN1571 monotherapy).

After the active dose is determined for AGEN1571 monotherapy (Study Treatment Arm 1), a 3+3 dose escalation design will be used to evaluate AGEN1571 (administered Q3W) in combination as follows with balstilimab and/or botensilimab. Patients will be assigned using a standard 3+3 dose escalation design for AGEN1571 in combination with balstilimab and/or botensilimab for Study Treatment Arms 2 to 4.

If treatment criteria are not met within the treatment window, the dose will be skipped, and the patient must not resume treatment until the next scheduled dose. Study treatment arms are as follows:

TABLE 5
Study Treatments

| Study Treatment Arms | Monotherapy or Combination Agents |
|---|---|
| Study Treatment Arm 1 | AGEN1571 monotherapy |
| | AGEN1571 will be administered on Day 1 and Day 22 of each 6-week cycle |
| | starting at 1 mg. |
| Study Treatment Arm 2 | 2-agent combination AGEN1571 + balstilimab |
| | The starting dose for AGEN1571 will be 1 dose level below the expected |
| | active dose (Arm 2, Cohort 1), followed by the expected active dose (Arm 2, |
| | Cohort 2) as cleared by the Safety Monitoring Committee (SMC) based on |
| | available safety, pharmacodynamic, and PK data from AGEN1571 |
| | monotherapy administration. |
| | Balstilimab at a fixed dose of 450 mg (Cohorts 1 and 2) will be administered |
| | following AGEN1571 administration on Day 1 and Day 22 of each 6-week |
| | cycle. |
| Study Treatment Arm 3 | 2-agent combination AGEN1571 + botensilimab |
| | The starting dose for AGEN1571 will be 1 dose level below expected active |
| | dose (Arm 3, Cohort 1), followed by the expected active dose (Arm 3, Cohort |
| | 2) as cleared by the SMC based on available safety, pharmacodynamic, and |
| | PK data from AGEN1571 monotherapy administration. |
| | AGEN1571 will be administered on Day 1 and Day 22 of each 6-week cycle |
| | followed by a dose of 150 mg botensilimab (Cohorts 1 and 2) administered |
| | on Day 1 of each 6-week cycle. |
| | Note: Lower doses (e.g., 50, 75, or 100 mg) of botensilimab can be used if |
| | deemed appropriate by SMC decision based on data from other studies. Use |
| | of a lower dose would be applied to all relevant cohorts. |
| Study Treatment Arm 4 | 3-agent combination AGEN1571 + balstilimab + botensilimab |
| | The starting dose for AGEN1571 will be 2 dose levels below the expected |
| | active dose (Cohort 1), followed by 1 dose level below the expected active |
| | dose (Cohort 2), followed by the expected active dose (Cohort 3) as cleared |
| | by the SMC based on available safety, pharmacodynamic, and PK data from |
| | AGEN1571 monotherapy administration. |
| | AGEN1571 in combination with a fixed dose of 450 mg balstilimab (Cohorts |
| | 1-3) will be administered on Day 1 and Day 22 of each 6-week cycle followed |
| | by a fixed 150 mg dose (Cohorts 1-3) of botensilimab administered on Day |
| | 1 of each 6-week cycle. |
| | Note: Lower doses (e.g., 50, 75, or 100 mg) of botensilimab can be used if |
| | deemed appropriate by SMC decision based on data from other studies. Use |
| | of a lower dose would be applied to all relevant cohorts. |

Patients diagnosed with advanced solid tumors will be assigned to cohorts by an Interactive Response Technology system. Alternate dosing schedules of AGEN1571 may also be explored depending on PK, pharmacodynamic, and safety results. For AGEN1571, the maximum recommended starting dose of 1 mg (approximately 0.014 mg/kg in a 70-kg patient) administered intravenously (IV) Q3W was derived from a 80% receptor occupancy (RO) approach as outlined by FDA scientists.

For Part 1 of the study, once an active dose of AGEN1571 is determined as either a single agent or in combination with balstilimab and/or botensilimab, an additional 15 evaluable patients may be added to that dose escalation cohort for any treatment arm (Study Treatment Arms 1 to 4) approved by the SMC to further evaluate safety, pharmacodynamic, and PK parameters.

Dose de-escalations are allowed between cohorts as cleared by the SMC on all study treatment arms. Intra-patient dose de-escalation or modification is not allowed.

### Part 2—Dose Expansion Phase for Specific Disease Indications

Based on data from Part 1 (dose escalation phase), and at the Sponsor's discretion, Part 2 of the study will open expansion cohorts by tumor type. Part 2 will enroll a minimum of 10 patients and a maximum of approximately 30 patients by tumor type at doses approved by the SMC for monotherapy or any combination therapy to further confirm the safety of a dose, to accommodate the need for additional PK data and/or additional pharmacodynamic data, to further evaluate any noted efficacy in the dose escalation phase that could comprise responses of long-term stable disease correlating with pharmacodynamic signals, and clinical activity in patients in doses previously cleared by the SMC.

All inclusion/exclusion criteria will be maintained for expansion cohorts. Expansion cohorts are subject to the same safety criteria outlined for the dose escalation cohorts. Specifically, no within-patient dose modifications are allowed.

### Number of Patients

For Part 1 (dose escalation phase) up to 98 DLT evaluable patients will be enrolled. For Part 2 (dose expansion phase), for each dose expansion cohort a minimum of 10 patients and a maximum of approximately 30 patients will be enrolled based on clinical considerations.

### Inclusion Criteria

In order to participate in the study, patient should meet all the following inclusion criteria:
1. Voluntarily agree to participate by giving signed, dated, and written informed consent prior to any study-specific procedures (participation in genetic testing is optional).
2. ≥18 years of age.
3. Histologically confirmed diagnosis of a solid tumor that is currently metastatic or locally advanced for which no standard therapy is available or standard therapy has failed.
4. Measurable disease on baseline imaging based on RECIST 1.1.
5. Life expectancy of at least 3 months.
6. ECOG performance status of 0 or 1.
7. Adequate organ and bone marrow reserve function, as indicated by the following laboratory values:
   a. Adequate hematological function, defined as absolute neutrophil count ≥1.5×10⁹/L, platelet count ≥100×10⁹/L, and hemoglobin ≥8 g/dL without recent transfusion (defined as a transfusion that has occurred within 2 weeks of the hemoglobin measurement).
   b. Adequate liver function, defined as serum albumin ≥3.0 g/dL, total bilirubin level ≤1.5× institutional upper limit of normal (IULN), aspartate aminotransferase ≤2.5×IULN, and alanine aminotransferase ≤2.5×IULN, and alkaline phosphatase ≤2.5×IULN or ≤5×ULN for patients with liver metastases.
   c. Adequate renal function defined as calculated creatinine clearance ≥40 mL/min as assessed by Cockcroft-Gault method.
   d. Adequate coagulation, defined as international normalized ratio or prothrombin time ≤1.5×IULN and activated partial thromboplastin time ≤1.5×IULN (unless patient is receiving anticoagulant therapy).
8. Patients with a history of prior malignancy are eligible if treatment was completed ≥2 years prior to the first dose of study treatment and the patient has no evidence of disease. Patients who have been maintained on stable doses of antineoplastic hormonal therapy within that 2-year time frame may also be eligible with approval of the Medical Monitor.
Patients with a history of prior early-stage basal/squamous cell skin cancer, or noninvasive or in situ cancers, who have undergone definitive treatment at any time are also eligible.
9. Patients must provide a sufficient and adequate formalin-fixed paraffin-embedded tumor tissue sample (biopsy) collected after the last dose of prior anti-cancer therapy and before the first dose of study treatment from a site not previously irradiated and agree to mandatory on-treatment biopsy if clinically feasible.
If a potential study participant cannot provide a tumor tissue sample as specified above, enrollment may be possible following discussion and approval from the Medical Monitor.
10. Female patients of childbearing potential must have a negative urine or serum pregnancy test at screening (within 72 hours of first dose of study medication) with repeat urine or serum pregnancy test of Day 1 of each cycle and at the end of treatment visit. If urine pregnancy test is positive, results must be confirmed with a serum pregnancy test. Non-childbearing potential is defined as:
   a. ≥50 years of age and has not had menses for greater than 1 year.
   b. Amenorrheic for ≥2 years without a hysterectomy and bilateral oophorectomy and a follicle-stimulating hormone value in the postmenopausal range upon pre-study (screening) evaluation.
   c. Status is post hysterectomy, bilateral oophorectomy, or tubal ligation.
Female patients who are diagnosed with a tumor that is known to secrete human chorionic gonadotropin must be certified not pregnant based on clinical evidence and investigator judgment.
Female patients of child-bearing potential must agree to use highly effective contraceptive measures starting with the screening visit through 90-Days after the last dose of study treatment. Highly effective contraception is defined in Appendix B, Guidance on Contraception, or as stipulated in national or local guidelines.
Note: Abstinence is acceptable if this is the established and preferred contraception for the patient.
11. Male patients with a female partner(s) of childbearing potential must agree to use highly effective contraceptive measures throughout the study starting with the screening visit through 90-Days after the last dose of study treatment is received. Males with pregnant partners must agree to use a condom; no additional method of contraception is required for the pregnant partner.
Note: Abstinence is acceptable if this is the established and preferred contraception for the patient.
12. Willing and able to comply with the requirements of the protocol.

### Exclusion Criteria

Patient will not be enrolled in the study, if any of the following criteria are met:
1. Currently participating and receiving study therapy or has participated in a study of an investigational agent and received study therapy or used an investigational device within 3 weeks of first dose of current study drug.
2. Received prior systemic cytotoxic chemotherapy, biological therapy, radiotherapy, or major surgery outside of the acceptable washout periods prior to first dose of study drug.
The following washout windows are acceptable from prior treatments, i.e., patients with time periods less than the following should be excluded:
   a. Cytotoxic agent or monoclonal antibody ≥3 weeks is acceptable (i.e., <3 weeks should be excluded).
   b. A 1-week washout is permitted for palliative radiation to non-central nervous system (CNS) disease.
   c. Small molecule targeted therapies and tyrosine kinase inhibitors ≥14 days or 5 half-lives is acceptable (i.e., <14 days or <5 half-lives should be excluded).
   d. Having a previous SARS-COV-2 vaccine >7 days before administration is acceptable. For vaccines requiring more than 1 dose, the full series should be completed prior to Cycle 1 Day 1, when feasible, and when the delay in initiation of study treatment would not put the study patients at risk.
   e. Prior radiation therapy to the CNS within 2 weeks before first treatment.
3. Persistent toxicity of NCI CTCAE Version 5.0 Grade ≥1 severity that is related to prior therapy.
4. Sensory neuropathy or alopecia of Grade≤2 are acceptable. Stable, compensated endocrinopathies (hypothyroidism, adrenal insufficiency, hypophysitis) that are the sequelae of immune-mediated adverse events >Grade 1 are acceptable. Known severe (Grade ≥3) hypersensitivity reactions to fully human monoclonal antibodies, or to any study drug excipients, or severe reaction to immuno-oncology agents, such as colitis or pneumonitis requiring treatment with steroids; or has a history of or active interstitial lung disease, any history of anaphylaxis, or uncontrolled asthma.
5. Patients with a condition requiring systemic treatment with either corticosteroids (>10 mg daily prednisone equivalent) within 14 days or any other immunosuppressive medication within 30-Days of the first dose of study treatment. Inhaled or topical steroids, and adrenal replacement steroid doses (≤10 mg daily prednisone equivalent), are permitted in the absence of active autoimmune disease.
6. Active CNS metastases.
Note: Patients are eligible if CNS metastases have been treated and patients are radiologically and clinically stable. Patients must have been either off corticosteroids, or on a stable or decreasing dose of ≤10 mg daily prednisone or equivalent for at least 2 weeks prior to the first dose of study treatment.
7. Active or history of autoimmune disease that requires systemic treatment within 2 years of the start of study drug (i.e., with use of disease-modifying agents, corticosteroids, or immunosuppressive drugs).
Note: Patients with autoimmune conditions requiring hormone replacement therapy or topical treatments are eligible.
8. Has had an allogeneic tissue/solid organ transplant, except for corneal transplants.
9. An active infection requiring treatment within 2 weeks of Cycle 1 Day 1 (C1D1), or active interstitial lung disease.
10. HIV positive.
   a. Patients with CD4>200 cells/mm³ are eligible.
   b. Patients with HIV viral load undetectable are eligible.
11. Active hepatitis B (HBV) or active hepatitis C (HCV).
   a. Patients with HBV infection are eligible if hepatitis B surface antigen and HBV DNA are negative.
   b. Patients with HCV infection are eligible if HCV RNA is negative.
12. Clinically significant (i.e., active) cardiovascular disease: cerebral vascular accident/stroke or myocardial infarction within 6 months of enrollment, unstable angina, congestive heart failure (New York Heart Association class ≥II), or serious uncontrolled cardiac arrhythmia requiring medication.
13. History or current evidence of any condition (including psychiatric or substance abuse disorder that would interfere with the requirements of the trial), therapy, any active infection requiring treatment within 2 weeks of CID1, or laboratory abnormality that might confound the results of the trial, interfere with the patient's participation for the full duration of the trial, or is not in the best interest of the patient to participate, in the opinion of the treating Investigator.
14. Legally incapacitated or has limited legal capacity.
15. Pregnant or breastfeeding.
16. QTc interval >480 msec at screening except if the prolonged QTc is due to right bundle branch block.
17. Uncontrolled hypertension and controlled hypertension (>140/90 mmHg) on more than 3 antihypertensive agents.

### Product, Dosage, and Mode of Administration

AGEN1571: Q3W (i.e., on Days 1 and 22 of each 6-week cycle) will be administered via IV infusion over 30 (+5) minutes.*

Balstilimab: Q3W (i.e., on Days 1 and 22 of each 6-week cycle) will be administered after AGEN1571 administration via IV infusion over 30 (+5) minutes.*

Botensilimab: Q6W (i.e., on Day 1 of each 6-week cycle) will be administered after AGEN1571 or balstilimab administration via IV infusion over 30 (+5) minutes.*

* All deviations from the infusion duration windows will be medically reviewed and the impact of the extension or reduction in infusion time will be assessed for clinical and research significance. If there is no impact on patient safety or any key study endpoints (e.g., PK data interpretation), these will not be regarded as protocol deviations at all, but as normal variations in clinical practice.

### Study Treatment Arm 1: AGEN1571 Monotherapy

AGEN1571 as a single agent will be evaluated at fixed doses using an accelerated titration design in 3 dose cohorts (1, 3, and 10 mg) in 1 to 6 patients. After completion of the accelerated titration phase, AGEN1571 as a single agent will be evaluated in a 3+3 dose escalation design in 4 dose cohorts (30 mg, 100 mg, 300 mg, and 1000 mg) of 3 to 6 patients per cohort Q3W (Day 1 and Day 22 of each 6-week cycle).

TABLE 6
Study Treatment Arm 1: AGEN1571 Monotherapy

| | | | Dose of AGEN1571 |
|---|---|---|---|
| Cohort^{a} | Patients (n) | Escalation Type | (mg) Q3W^{b} |
| 1 | 1-6 | 1 + 2 + 3 Accelerated | 1 |
| 2 | 1-6 | 1 + 2 + 3 Accelerated | 3 |
| 3 | 1-6 | 1 + 2 + 3 Accelerated | 10 |
| 4 | 3-6 | 3 + 3 | 30 |
| 5 | 3-6 | 3 + 3 | 100 |
| 6 | 3-6 | 3 + 3 | 300 |
| 7 | 3-6 | 3 + 3 | 1000 |

Abbreviations: Q3W = every 3 weeks.
^{a}Cohorts 1 to 3 will follow an accelerated titration design for dose escalation. Cohorts 4 to 7 will follow a 3 + 3 design for dose escalation.
^{b}Based on the totality of safety data, planned dose levels of AGEN1571 monotherapy may be reduced, or intermediate dose levels may be added after discussion and agreement between the Sponsor and Investigators.

Note: Alternate dosing schedules of AGEN1571 may be explored depending on safety, preliminary efficacy, PK, and pharmacodynamic data.

Note: Following the identification of an active dose in Study Treatment Arm 1, up to an additional 15 evaluable patients may be enrolled into the active dose cohort and treated from a safety/PK/pharmacodynamic perspective.

### Study Treatment Arm 2: AGEN1571+Balstilimab

AGEN1571 in combination with balstilimab will be evaluated in 2 dose cohorts using a 3+3 dose escalation design.

TABLE 7
Study Treatment Arm 2: AGEN1571 + balstilimab

| | | | | Dose of |
|---|---|---|---|---|
| | Patients | Escalation | Dose of | Balstilimab |
| Cohort | (n) | Type | AGEN1571 Q3W | (mg) Q3W |
| 1 | 3-6 | 3 + 3 | 1 dose level below | 450 |
| | | | expected active dose | |
| 2 | 3-6 | 3 + 3 | Expected active dose | 450 |

Abbreviations: DLT = dose-limiting toxicity; PK = pharmacokinetic; Q3W = every 3 weeks; SMC = Safety Monitoring Committee.

Note: For the combination, AGEN1571 should be administered before balstilimab with 30-minute interval between the infusion.

Note: Alternate dosing schedules of AGEN1571 and balstilimab may be explored depending on safety, preliminary efficacy, PK, and pharmacodynamic data.

Note: Following the identification of an active dose, up to an additional 15 evaluable patients may be treated at the active dose for AGEN1571+balstilimab from a safety/PK/pharmacodynamic perspective.

### Study Treatment Arm 3: AGEN1571+Botensilimab

AGEN1571 in combination with botensilimab will be evaluated in 2 dose cohorts using a 3+3 dose escalation design.

TABLE 8
Study Treatment Arm 3: AGEN1571 + botensilimab

| | | | | Dose of |
|---|---|---|---|---|
| | Patients | Escalation | Dose of | Botensilimab |
| Cohort | (n) | Type | AGEN1571 Q3W | (mg) Q6W* |
| 1 | 3-6 | 3 + 3 | 1 dose level below | 150* |
| | | | expected active dose | |
| 2 | 3-6 | 3 + 3 | Expected active dose | 150* |

Abbreviations: DLT = dose-limiting toxicity; PK = pharmacokinetic; Q3W = every 3 weeks; Q6W = every 6 weeks; SMC = Safety Monitoring Committee.
*Note:
Lower doses (e.g., 50, 75, or 100 mg) of botensilimab can be used if deemed appropriate by SMC decision based on data from other studies. Use of a lower dose would be applied to all relevant cohorts.

Note: For the combination, AGEN1571 should be administered before botensilimab with 30-minute interval between the infusion.

Note: Alternate dosing schedules of AGEN1571 and botensilimab may be explored depending on safety, preliminary efficacy, PK, and pharmacodynamic data.

Note: Following the identification of an active dose, up to an additional 15 evaluable patients may be treated at the active dose for AGEN1571+botensilimab from a safety/PK/pharmacodynamic perspective.

### Study Treatment Arm 4: AGEN1571+balstilimab+botensilimab

AGEN1571 in combination with balstilimab and botensilimab will be evaluated in 2 dose cohorts using a 3+3 dose escalation design.

TABLE 9
Study Treatment Arm 4: AGEN1571 + balstilimab + botensilimab

| | | Esca- | Dose of | Dose of | Dose of |
|---|---|---|---|---|---|
| | Patients | lation | AGEN1571 | Balstilimab | Botensilimab |
| Cohort | (n) | Type | Q3W | (mg) Q3W | (mg) Q6W* |
| 1 | 3-6 | 3 + 3 | 2 dose levels | 450 | 150* |
| | | | below expected | | |
| | | | active dose | | |
| 2 | 3-6 | 3 + 3 | 1 dose level | 450 | 150* |
| | | | below expected | | |
| | | | active dose | | |
| 3 | 3-6 | 3 + 3 | Expected | 450 | 150* |
| | | | active dose | | |

Abbreviations: DLT = dose-limiting toxicity; PK = pharmacokinetic; Q3W = every 3 weeks; Q6W = every 6 weeks; SMC = Safety Monitoring Committee.
*Note:
Lower doses (e.g., 50, 75, or 100 mg) of botensilimab can be used if deemed appropriate by SMC decision based on data from other studies. Use of a lower dose would be applied to all relevant cohorts.

Note: For the combination, AGEN1571 should be administered before balstilimab with a 30-minute interval between infusion, followed by botensilimab with 30-minute interval after infusion with balstilimab.

Note: Alternate dosing schedules may be explored depending on safety, preliminary efficacy, PK, and pharmacodynamic data.

Note: Following the identification of an active dose, up to an additional 15 evaluable patients may be treated at the active dose for AGEN1571+balstilimab+botensilimab from a safety/PK/pharmacodynamic perspective.

### Duration of Treatment

Treatment with AGEN1571 monotherapy or AGEN1571 in combination with balstilimab and/or botensilimab will continue until confirmed progressive disease, unacceptable toxicity, or up to 2 years, whichever occurs first. After treatment discontinuation, patient will be followed for safety at 30 (±7) days and 90 (±7) days, and for long-term follow-up every 2 months for at least 12 months from last administration.

### Criteria for Evaluation

Safety: Safety will be assessed by evaluating AEs graded according to NCI CTCAE Version 5.0, vital signs (temperature, pulse, respiratory rate, and blood pressure), physical examinations, 12-lead ECG, ECOG performance status, and clinical laboratory assessments. DLT will be assessed during the dose escalation phase. An SMC will be established to assess safety, decide on dose escalation, and define the RP2D.

Pharmacokinetics: Serum AGEN1571 PK parameters may include (but are not limited to) maximum observed drug concentration at steady state, minimum observed drug concentration at steady state, area under the drug concentration-time curve within time span t1 to t2 at steady state, area under the drug concentration-time curve from time zero to time t, area under the drug concentration-time curve from time zero to infinity, time to maximum observed drug concentration, elimination rate constant, terminal elimination half-life, systemic drug clearance, and volume of distribution. Both noncompartmental analysis and compartmental modeling (e.g., population pharmacokinetics [PopPK]) will be used to analyze PK. Additional PK exposure metrics for serum balstilimab and botensilimab will be assessed via PopPK.

Immunogenicity: Serum ADAs will be assessed to determine the potential impact on PK exposure metrics and biological activity for AGEN1571, balstilimab, and botensilimab. ADA positive samples will be assessed for antibody neutralization.

Pharmacodynamics: Plasma, serum, whole blood samples, and tumor biopsies will be collected for exploratory biomarker analysis.

Efficacy: Response assessments will be performed according to RECIST 1.1. On-study imaging will be performed every 9 weeks (±7 days) after initiation of treatment for 12 months, and every 12 weeks (±7 days) thereafter.

### Statistical Methods

The total sample size in Part 1 is expected to be up to 98 evaluable treated patients. Since the study uses a 3+3 design, sample size will be determined by the number of evaluated dose levels. Sample size in Part 2 will depend on the number of expansion cohorts recommended by SMC (10-30 patients per cohort).

Efficacy endpoints will be based upon RECIST v1.1. The following efficacy endpoints will be analyzed and summarized in order to evaluate the antitumor activities of all combinations: ORR, DOR, DCR, PFS and OS. PFS, DOR and OS will be estimated using the Kaplan-Meier method. Waterfall plots of maximum tumor shrinkage per patient will be presented.

Safety will be determined by the spontaneous reporting of AEs and by laboratory values (hematology, clinical chemistry, coagulation, and urinalysis). Vital signs, physical examinations, and electrocardiogram findings will also be used in determining the safety profile. The severity of AEs will be graded according to the NCI-CTCAE version 5.0. The incidence of DLT events and TEAEs will be reported as the number (percentage) of patients with TEAEs by system organ class and preferred term. Descriptive statistics will be used in general to summarize study results, i.e., statistics for continuous variables will include mean, standard deviation, median, minimum and maximum, and for the PK summary, the coefficient of variation and geometric means will also be included. Qualitative variables will be summarized by counts and percentages. Unless otherwise specified, calculation of proportions will be based on the sample size of the analysis set of interest.

Interim analyses will be conducted as appropriate to assess preliminary safety and evidence of activity. They will be exploratory in nature and for descriptive purpose only without formal statistical testing.

### Example 2: AGEN1571 Combined with PD-1 Antagonist Balstilimab Potentiates T Cell Activation, NK Cell Activation, and NKT Cell Activation

The combination potential between AGEN1571 and an anti-PD-1 antagonist (balstilimab; AGEN2034), was assessed in an immune cell activation assay where allorecognition of tumor cells promoted the activity of T cells, NK cells, and NKT cells. Cells from a human choriocarcinoma JEG3 cell line that endogenously expresses HLA-G (Avril et al., J Immunol (1999) 162 (10): 5902-5909.) were used as target cells. Peripheral blood mononuclear cells (PBMCs) from healthy donors were used as effector cells. Cryopreserved peripheral blood mononuclear cells (PBMCs) from 10 healthy donors were primed overnight with IL-2 and IL-15 in the presence of AGEN1571, AGEN2034, or isotype control antibodies. On the next day, JEG-3^{HLA-A2} cells expressing HLA-G and HLA-A2 were added to the primed PBMCs (1:40 tumor cell: PBMC ratio) and co-cultured for 72h. Immune cell activation was assessed on the basis of increased CD25 expression on T cells (CD3⁺CD4⁺ and CD3⁺CD8⁺), NK cells (CD3⁻ CD56⁺), and NKT cells (CD3⁺CD56⁺) detected by flow cytometry. Cytokine secretion in the cultured supernatants was analyzed by an assessment of cytokine levels tested by a bead-based immunoassay (Luminex).

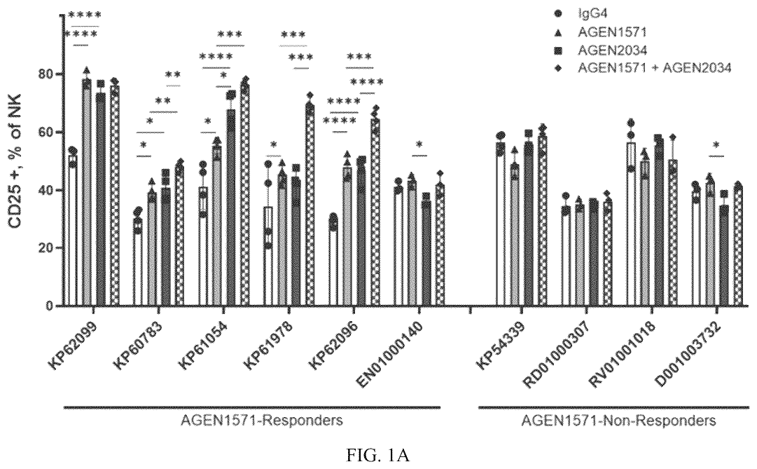

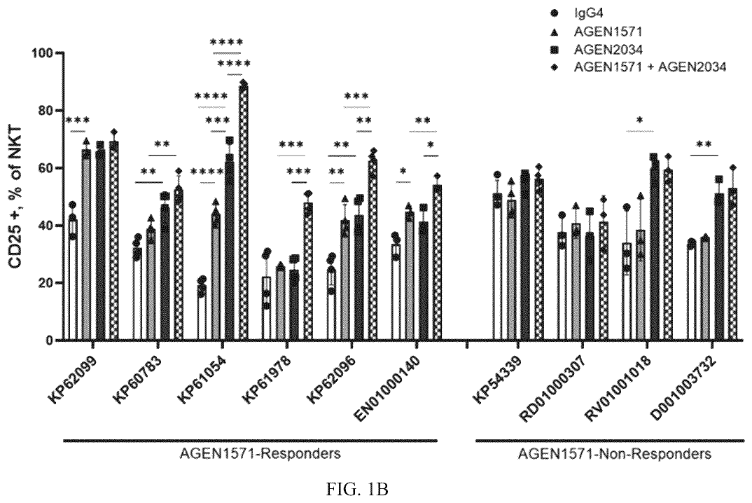

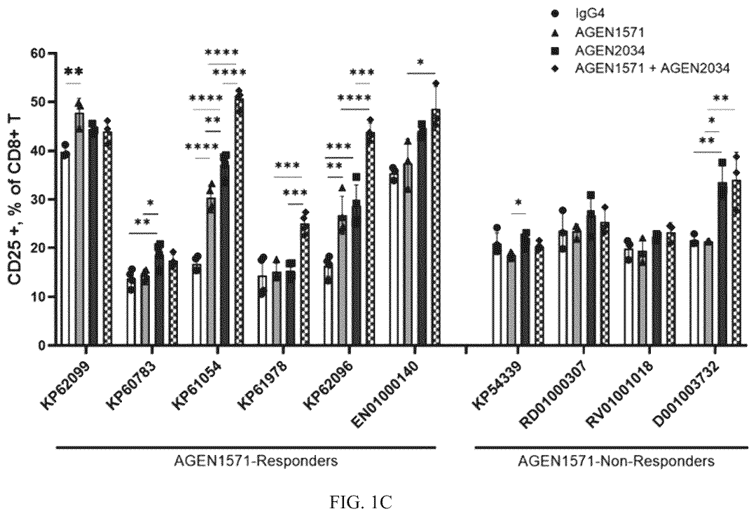

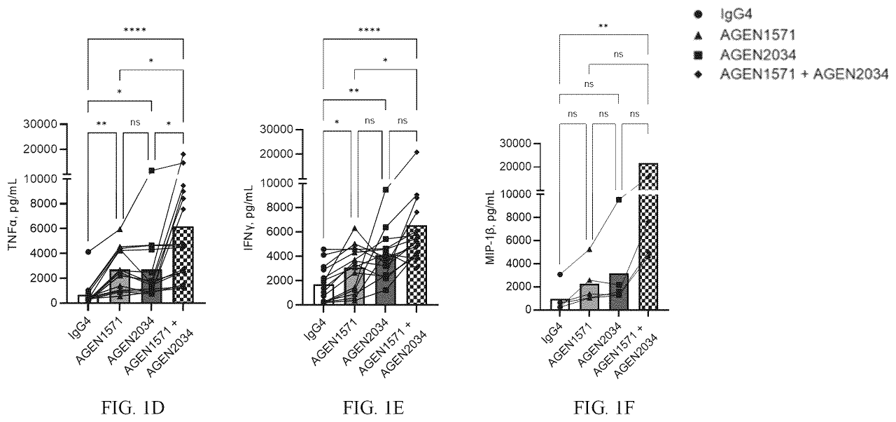

AGEN1571-mediated ILT2 blockade enhanced CD25 expression on CD8⁺ T, NK (CD3-CD56⁺), and NKT (CD3⁺CD56⁺) cells, and combination with a PD-1 inhibitor (balstilimab; AGEN2034) further enhanced activation of NK, NKT, and cytotoxic CD8+ T cells. As demonstrated in FIGS. 1A-C, an additive effect of ILT2 (AGEN1571) with PD-1 (balstilimab; AGEN2034) co-blockade was observed in 5 of 6 donors responsive to AGEN1571 treatment as assessed by augmented induction of CD25 expression on NKT cells. In addition, as shown in FIG. 1D, AGEN1571 combined with AGEN2034 enhanced TNF alpha, IFN gamma, and IL-1 beta cytokine secretion. These data demonstrate the potential therapeutic utility of combining AGEN1571 and a PD-1 inhibitor (AGEN2034) in a cancer treatment setting.

### Example 3: AGEN1571 Promotes FcγR NFAT Signaling Induced by AGEN1181

The ability for AGEN1571 to enhance FcγR signaling induced by an Fc enhanced IgG1k human monoclonal antibody that selectively binds to human anti-cytotoxic T-lymphocyte-associated antigen 4 (CTLA-4) (AGEN1181) was tested. A Jurkat T cell line engineered to express (i) human ILT2, (ii) FcγRIIIA receptor (CD16), and (iii) a luciferase reporter gene induced by a nuclear factor of activated T-cells (NFAT) response element (Jurkat ILT2 CD16 NFAT luciferase) was co-cultured with a Raji B cell line endogenously expressing ILT2 ligands and engineered to express CTLA-4 (Raji CTLA-4).

Jurkat ILT2 CD16 NFAT luciferase cells were co-cultured with Raji CTLA-4 cells in the presence of 0.01-10 μg/mL of AGEN1181 and 10-4-5 μg/mL of AGEN1571 or a corresponding IgG₄ isotype control antibody. AGEN1181 opsonization of Raji CTLA-4-expressing cells induces CD16 signaling which is then inhibited by engagement of ILT2 ligand(s). Without wishing to be bound by any particular theory, it is thought that blockade of ILT2 ligand interactions by AGEN1571 relieves ILT2-mediated inhibition of AGEN1181-induced CD16 signaling to enhance luciferase expression. After overnight incubation, luciferase activity was measured as an endpoint to evaluate FcγR signaling.

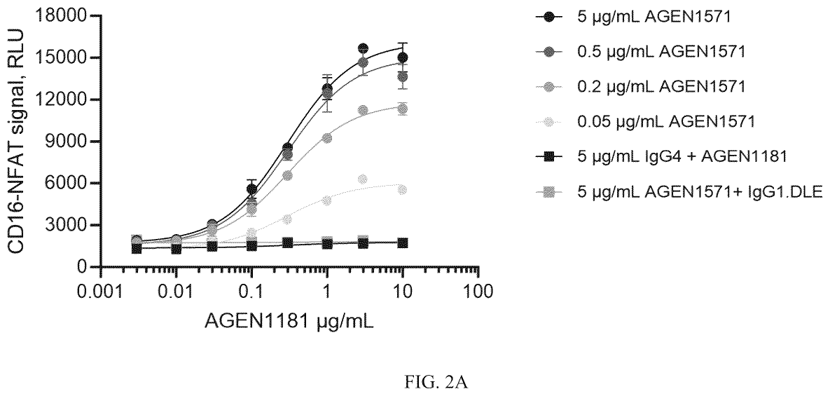

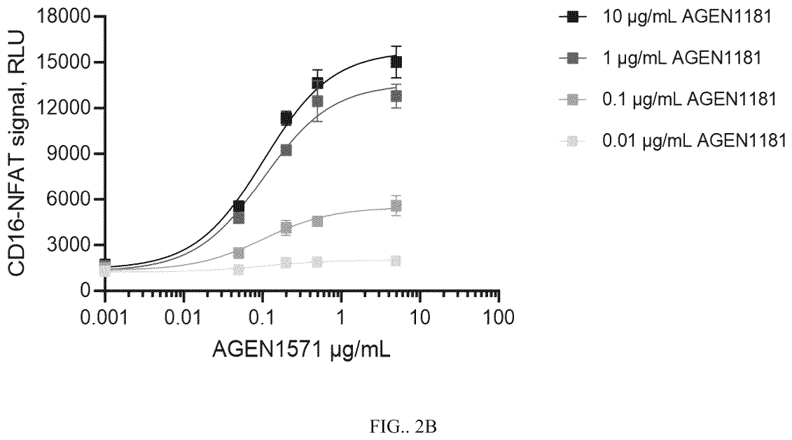

As demonstrated in FIGS. 2A and 2B, AGEN1571 enhanced AGEN1181-induced FcγR stimulation in a dose dependent manner. The isotype control antibody did not increase the expression of the reporter gene even at the highest tested concentration of 10 μg/mL. In the absence of AGEN1571, AGEN1181 induced negligible FcγR signaling (luciferase), indicating that ILT2 is a potent antagonist of CD16-NFAT signaling. No activation of the reporter gene was observed with the Fc enhanced isotype antibody in the presence of AGEN1571. These data demonstrate the potential therapeutic utility of combining AGEN1571 and AGEN1181 in a cancer treatment setting.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes. Other embodiments are within the following claims.

1. A method of treating cancer in a subject in need thereof, the method comprising administering to the subject a first antibody that specifically binds to human ILT2 at a dose of about 1 mg to about 2000 mg, wherein the first antibody comprises: a heavy chain variable region (VH) comprising the CDRH1, CDRH2, and CDRH3 amino acid sequences of the VH amino acid sequence set forth in SEQ ID NO: 7; and a light chain variable region (VL) comprising the CDRL1, CDRL2, and CDRL3 amino acid sequences of the VL amino acid sequence set forth in SEQ ID NO: 8.

2. A method of treating cancer in a subject in need thereof, the method comprising administering to the subject:
(a) a first antibody that specifically binds to human ILT2, wherein the antibody comprises: a VH comprising the CDRH1, CDRH2, and CDRH3 amino acid sequences of the VH amino acid sequence set forth in SEQ ID NO: 7; and a VL comprising the CDRL1, CDRL2, and CDRL3 amino acid sequences of the VL amino acid sequence set forth in SEQ ID NO: 8; and
(b) (i) a human PD-1 inhibitor, a human CTLA-4 inhibitor, a human PD-L1 inhibitor, or a human PD-L2 inhibitor; or (ii) a human PD-1 inhibitor; and a human CTLA-4 inhibitor.

3.-6. (canceled)

7. The method of claim 1, wherein the first antibody is administered at a dose of about 1 mg to about 1000 mg, optionally wherein the first antibody is administered at a dose of about 1 mg, about 3 mg, about 10 mg, about 30 mg, about 100 mg, about 300 mg, or about 1000 mg, optionally wherein:
the first antibody is administered intravenously, and/or the first antibody is administered by intravenous infusion over about 30 minutes.

8.-10. (canceled)

11. The method of claim 1, wherein;
the first antibody is administered once weekly, once every 2 weeks, once every 3 weeks, once every 4 weeks, once every 5 weeks, or once every 6 weeks;
the first antibody is administered intravenously at a dose of 1 mg once every 3 weeks;
the first antibody is administered intravenously at a dose of 3 mg once every 3 weeks;
the first antibody is administered intravenously at a dose of 10 mg once every 3 weeks;
the first antibody is administered intravenously at a dose of 30 mg once every 3 weeks;
the first antibody is administered intravenously at a dose of 100 mg once every 3 weeks;
the first antibody is administered intravenously at a dose of 300 mg once every 3 weeks; and/or
the first antibody is administered intravenously at a dose of 1000 mg once every 3 weeks.

12.-23. (canceled)

24. The method of claim 1, wherein the cancer is a solid tumor, optionally wherein the solid tumor is metastatic or locally advanced.

25. (canceled)

26. The method of claim 1, wherein the cancer is selected from the group consisting of bladder cancer, brain cancer, breast cancer, cervical cancer, colon and/or rectal cancer, endometrial cancer, head and neck cancer, kidney cancer, liver cancer, melanoma, mesothelioma, non-small cell lung cancer, oral cancer, ovarian cancer, pancreatic cancer, prostate cancer, sarcoma, skin cancer, small cell lung cancer, stomach cancer, and thyroid cancer.

27. The method of claim 1, wherein before administration of the first antibody:
(a) the subject is at least 18 years of age;
(b) the subject has a histologically confirmed diagnosis of a solid tumor that is currently metastatic or locally advanced, wherein no standard therapy is available or standard therapy has failed;
(c) the subject has measurable disease on baseline imaging as assessed according to Response Evaluation Criteria in Solid Tumors Version 1.1 (RECIST 1.1);
(d) the subject has a predicted life expectancy of ≥3 months;
(e) the subject has an Eastern Cooperative Oncology Group performance status of 0-1;
(f) the subject has adequate organ and bone marrow reserve function as defined by one or more of:
(i) absolute neutrophil count ≥1.5×10⁹/L;
(ii) platelet count ≥100×10⁹/L;
(iii) hemoglobin ≥8.0 g/dL without transfusion within 2 weeks prior to hemoglobin measurement;
(iv) serum albumin ≥3.0 g/dL;
(v) total bilirubin ≤1.5× upper limit of normal (ULN);
(vi) aspartate aminotransferase ≤2.5×ULN;
(vii) alanine aminotransferase ≤2.5×ULN;
(viii) alkaline phosphatase ≤2.5×ULN, or ≤5.0×ULN for subjects with liver metastases;
(ix) creatinine clearance ≥40 mL/min as assessed by Cockcroft-Gault method; and/or
(x) international normalized ratio or prothrombin time ≤1.5×ULN and activated partial thromboplastin time ≤1.5×ULN, unless patient is receiving anticoagulant therapy;
(g) the subject is not pregnant and/or breastfeeding;
(h) the subject has not had a cytotoxic therapy or other monoclonal antibody therapy within 3 weeks prior;
(i) the subject has not had radiation therapy to the central nervous system (CNS) within 2 weeks prior or non-CNS radiation therapy within one week prior; and/or
(j) the subject has not had small molecule targeted therapy or tyrosine kinase inhibitor therapy within 2 weeks prior or 5 half-lives;
(k) the subject does not have any persistent toxicities (Common Terminology Criteria for Adverse Events [CTCAE]≥1) from prior cancer therapies;
(l) the subject does not have sensory neuropathy or alopecia of Grade ≥2;
(m) the subject does not have a history of or active interstitial lung disease;
(n) the subject does not have a history of anaphylaxis or uncontrolled asthma;
(o) the subject does not have a condition requiring systemic treatment with either corticosteroids (>10 mg daily prednisone equivalent) within 2 weeks prior or other immunosuppressive medication within 30 days prior;
(p) the subject does not have active CNS metastases, unless the CNS metastases has been treated and the subject is radiologically and clinically stable;
(q) the subject does not have active or history of autoimmune disease that requires systemic treatment within 2 years prior, unless treatment consists of hormone replacement therapy or topical treatment;
(r) the subject has not had an allogeneic tissue or solid organ transplant, except for a corneal transplant;
(s) the subject has not had an active infection requiring treatment within 2 weeks prior;
(t) the subject is not HIV positive, unless the subject has CD4>200 cells/mm³ or an undetectable viral load;
(u) the subject does not have active hepatitis B (HBV) or hepatitis C (HPC), unless hepatitis B surface antigen and HBV DNA, or HCV RNA, respectively, are negative;
(v) the subject does not have clinically significant cardiovascular disease, as defined by one or more of:
(i) cerebral vascular accident or stroke or myocardial infarction within 6 months prior;
(ii) unstable angina;
(iii) congestive heart failure (New York Heart Association class ≥II); and/or
(iv) uncontrolled cardiac arrhythmia requiring medication;
(w) the subject does not have QTc interval >480 msec, unless prolonged QTc is due to right bundle branch block; and/or
(x) the subject does not have uncontrolled hypertension (>140/90 mmHg), or controlled hypertension on more than 3 antihypertensive agents.

28. The method of claim 1, wherein the first antibody comprises:
the CDRH1, CDRH2, CDRH3, CDRL1, CDRL2, and CDRL3 amino acid sequences, set forth in SEQ ID NOs: 1, 2, 3, 4, 5, and 6, respectively;
the VH amino acid sequence of SEQ ID NO: 7, and/or the VL amino acid sequence of SEQ ID NO: 8;
the VH and VL amino acid sequences of SEQ ID NOs: 7 and 8, respectively;
a heavy chain constant region selected from the group consisting of human IgG₁, IgG₂, IgG₃, IgG₄, IgA₁, IgA₂, and IgM, optionally wherein the heavy chain constant region is a human IgG₄ heavy chain constant region comprising a P at position 228, numbered according to the EU numbering system;
a heavy chain comprising the amino acid sequence of SEQ ID NO: 9, and/or a light chain comprising the amino acid sequence of SEQ ID NO: 10;
a heavy chain and a light chain comprising the amino acid sequences of SEQ ID NOs: 9 and 10, respectively.

29.-34. (canceled)

35. The method of claim 2, wherein
the human PD-1 inhibitor is a second antibody that specifically binds to human PD-1, human PD-L1, or human PD-L2.

36. The method of claim 35, wherein the second antibody comprises:
a VH comprising the CDRH1, CDRH2, and CDRH3 amino acid sequences of the VH amino acid sequence set forth in SEQ ID NO: 17; and a VL comprising the CDRL1, CDRL2, and CDRL3 amino acid sequences of the VL amino acid sequence set forth in SEQ ID NO: 18;
the CDRH1, CDRH2, CDRH3, CDRL1, CDRL2, and CDRL3 amino acid sequences, set forth in SEQ ID NOs: 11, 12, 13, 14, 15, and 16, respectively;
the second antibody comprises the VH amino acid sequence of SEQ ID NO: 17, and/or the VL amino acid sequence of SEQ ID NO: 18;
the second antibody comprises the VH and VL amino acid sequences of SEQ ID NOs: 17 and 18, respectively;
the second antibody comprises a heavy chain constant region selected from the group consisting of human IgG₁, IgG₂, IgG₃, IgG₄, IgA₁, IgA₂, and IgM, optionally wherein the heavy chain constant region is a human IgG₄ heavy chain constant region comprising a P at position 228, numbered according to the EU numbering system;
the second antibody comprises a heavy chain comprising the amino acid sequence of SEQ ID NO: 19, and/or a light chain comprising the amino acid sequence of SEQ ID NO: 20;
the second antibody comprises a heavy chain and a light chain comprising the amino acid sequences of SEQ ID NOs: 19 and 20, respectively; and/or
the second antibody is selected from the group consisting of balstilimab, adebrelimab, atezolizumab, avelumab, camrelizumab, cemiplimab, cosibelimab, dostarlimab, durvalumab, enlonstobart, envafolimab, nivolumab, pembrolizumab, penpulimab, pidilizumab, prolgolimab, pucotenlimab, retifanlimab, serplulimab, sintilimab, socazolimab, sugemalimab, tagitanlimab, tislelizumab, toripalimab, and zimberelimab.

37.-44. (canceled)

45. The method of claim 35, wherein the second antibody:
is administered at a dose of about 450 mg;
is administered at a dose of about 3 mg/kg;
is administered by intravenous infusion over about 30 minutes;
is administered once weekly, once every 2 weeks, or once every 3 weeks; and/or
is administered to the subject subsequent to administration of the first antibody.

46.-51. (canceled)

52. The method of claim 2, wherein the human CTLA-4 inhibitor is a third antibody that specifically binds human CTLA-4.

53. The method of claim 52, wherein the third antibody comprises:
a VH comprising the CDRH1, CDRH2, and CDRH3 amino acid sequences of the VH amino acid sequence set forth in SEQ ID NO: 27; and a VL comprising the CDRL1, CDRL2, and CDRL3 amino acid sequences of the VL amino acid sequence set forth in SEQ ID NO: 28;
the CDRH1, CDRH2, CDRH3, CDRL1, CDRL2, and CDRL3 amino acid sequences, set forth in SEQ ID NOs: 21, 22, 23, 24, 25, and 26, respectively;
the VH amino acid sequence of SEQ ID NO: 27, and/or the VL amino acid sequence of SEQ ID NO: 28;
the third antibody comprises the VH and VL amino acid sequences of SEQ ID NOs: 27 and 28, respectively;
the third antibody comprises a heavy chain constant region selected from the group consisting of human IgG₁, IgG₂, IgG₃, IgG₄, IgA₁, IgA₂, and IgM, optionally wherein the heavy chain constant region is a human IgG heavy chain constant region comprising a D at position 239, a L at position 330, and an E at position 332, numbered according to the EU numbering system;
the third antibody comprises a human IgG₁ heavy chain constant region that is a variant of a wild type human IgG₁ heavy chain constant region, wherein the variant human IgG₁ heavy chain constant region binds to FcγRIIIA with a higher affinity as compared to the affinity that the wild type human IgG₁ heavy chain constant region binds to FcγRIIIA, optionally wherein the heavy chain constant region is a human IgG₁ heavy chain constant region comprising a D at position 239, a L at position 330, and an E at position 332, numbered according to the EU numbering system;
the third antibody is afucosylated;
the third antibody comprises a heavy chain comprising the amino acid sequence of SEQ ID NO: 29, and/or a light chain comprising the amino acid sequence of SEQ ID NO: 30;
the third antibody comprises a heavy chain and a light chain comprising the amino acid sequences of SEQ ID NOs: 29 and 30, respectively; and/or
the third antibody is selected from the group consisting of botensilimab, ipilimumab, and tremelimumab.

54.-63. (canceled)

64. The method of claim 52, wherein the third antibody:
is administered at a dose of about 150 mg, 75 mg, or 50 mg;
is administered at a dose of about 2 mg/kg or about 4 mg/kg;
is administered by intravenous infusion over about 30 minutes;
is administered once every 2 weeks, once every 3 weeks, once every 4 weeks, or once every 6 weeks.

65.-73. (canceled)

74. The method of claim 52, wherein the third antibody is administered to the subject subsequent to administration of the first antibody, or subsequent to administration of the first antibody and second antibody.

75. (canceled)

76. The method of claim 2, wherein;
the human PD-1 inhibitor comprises an inhibitory small molecule, an inhibitory peptide, or inhibitory Fc fusion protein;
the human CTLA-4 inhibitor comprises an inhibitory small molecule, an inhibitory peptide, or inhibitory Fc fusion protein;
the human PD-L1 inhibitor comprises an antagonist antibody or fragment thereof, an inhibitory small molecule, an inhibitory peptide, or inhibitory Fc fusion protein; or
the human PD-L2 inhibitor comprises an antagonist antibody or fragment thereof, an inhibitory small molecule, an inhibitory peptide, or inhibitory Fc fusion protein.

77.-79. (canceled)

80. A method of treating cancer in a subject in need thereof, the method comprising administering to the subject:
(a) a first antibody that specifically binds to human ILT2, wherein the antibody comprises a heavy chain and a light chain comprising the amino acid sequences of SEQ ID NOs: 9 and 10, respectively;
(b) balstilimab at a dose of 450 mg once every 3 weeks by intravenous infusion; and
(c) botensilimab at a dose of 150 mg once every 6 weeks by intravenous infusion.

81. The method of claim 80, wherein the first antibody:
is administered at a dose of about 1 mg to about 1000 mg;
is administered at a dose of about 1 mg, about 3 mg, about 10 mg, about 30 mg, about 100 mg, about 300 mg, or about 1000 mg;
is administered intravenously;
is administered by intravenous infusion over about 30 minutes;
is administered once weekly, once every 2 weeks, once every 3 weeks, once every 4 weeks, once every 5 weeks, or once every 6 weeks; and/or
is administered intravenously at a dose of 1 mg once every 3 weeks, 3 mg once every 3 weeks, 10 mg once every 3 weeks, 30 mg once every 3 weeks, 100 mg once every 3 weeks, 300 mg once every 3 weeks, or 1000 mg once every 3 weeks.

82.-97. (canceled)

98. The method of claim 80, wherein the cancer is a solid tumor, optionally wherein the solid tumor is metastatic or locally advanced.

99. (canceled)

100. The method of claim 98, wherein the cancer is selected from the group consisting of bladder cancer, brain cancer, breast cancer, cervical cancer, colon and/or rectal cancer, endometrial cancer, head and neck cancer, kidney cancer, liver cancer, melanoma, mesothelioma, non-small cell lung cancer, oral cancer, ovarian cancer, pancreatic cancer, prostate cancer, sarcoma, skin cancer, small cell lung cancer, stomach cancer, and thyroid cancer.

101.-103. (canceled)
